(19) United States
(12) Patent Application Publication
(10) Pub. No.: US 2009/0156471 A1
(43) Pub. Date: Jun. 18, 2009

(54) Use of anti-amyloid agents for treating and typing pathogen infections

(75) Inventors: Ehud Gazit, Ramat-HaSharon (IL); Izhack Cherny, Tel-Aviv (IL)

Correspondence Address: MARTIN D. MOYNIHAN d/b/a PRTSI, INC., P.O. BOX 16446, ARLINGTON, VA, 22215, US

(73) Assignee: RAMOT AT TEL AVIV UNIVERSITY LTD., TEL-AVIV (IL)

(21) Appl. No.: 11/632,410

(22) PCT Filed: Jul. 14, 2005
(86) PCT No.: PCT/IL05/00754

§ 371 Date: Jan. 16, 2007

(51) Int. Cl. A61K 38/10; A61K 38/08; C12Q 1/02; A61K 38/06; A61K 38/07; A61K 38/05; A61K 31/404; A61K 31/352; A61K 31/465

(52) U.S. Cl. 514/9; 514/16; 514/15; 435/29; 514/14; 514/17; 514/18; 514/19; 514/415; 514/456; 514/343

(57) ABSTRACT

A method of preventing or treating a pathogen infection in a subject is provided. The method comprising administering to a subject in need thereof a therapeutically effective amount of an anti amyloid agent, thereby treating or preventing the pathogen infection in the subject.

### FIELD AND BACKGROUND OF THE INVENTION

The present invention relates to methods and compositions for the treatment of pathogen infection, such as bacterial infection.

A biofilm is a structured community of bacterial cells encapsulated in a polymeric matrix and adherent to an inert or living surface. It is estimated that virtually all bacteria in nature attach to a surface in the form of a biofilm [Murphy et al (2002) BMC Microbiology 2002, 2:7]. It has been shown that bacteria growing in biofilms can become up to one thousand fold more resistant to antibiotics and to other biocides compared with their planktonic counterparts. As a result of this increased resistance, biofilm infections are unable to be treated effectively with conventional antibiotic therapy.

Various hypotheses have been formulated to explain the reduced susceptibility of biofilms to antibiotics. One hypothesis suggests that only the surface layers of a biofilm are exposed to a lethal dose of the antibiotic due to a reaction-diffusion barrier which limits transport of the antibiotic into the biofilm [Hoyle, B. D. et al., 1992, Antimicrob. Agents Chemother. 36:2054-2056]. Synthesis of an antibiotic-degrading enzyme, such as a β-lactamase enzyme [Giwercman, B., E. T. et al., 1991, Antimicrob. Agents Chemother. 35:1008-1010], or sorption of an antibiotic to biofilm components [Stewart, P. S., 1996, Antimicrob. Agents Chemother. 40:2517-2522] could also give rise to such a situation. In that context, the binding and sequestering action of periplasmic glucans have recently been suggested as a possible mechanism for biofilm-specific resistance [Thien-Fah Mah et al., Nature, 426:306-310, Nov. 20, 2003]. It has also been proposed that some microorganisms within the biofilm exist in a more recalcitrant phenotypic state. The roles of various physiological factors, such as growth rate, biofilm age, and starvation, have been examined.

As well as increasing antibiotic resistance, biofilms also show an increased resistance to clearance by the immune system [Meluleni et al. (1995) J. Immunol. 155:2029-2038].

Colonization of bacteria on medical and dental devices (such as prosthetic devices, contact lenses, feeding tubes, pacemakers, artificial joints, heart valve replacements and other surgical and dental implants) is a major cause of chronic infection and device failure. Moreover, biofilm formation results in the antibiotic resistance of bacteria in cystic fibrosis and immunocompromised patients. Other studies have adduced evidence suggesting the involvement of bacterial biofilms in respiratory tract infections and otitis media [Murphy et al (2002) BMC Microbiology 2002, 2:7].

However, currently there are no drugs that specifically target the assembly events that lead to the formation of the biofilm. There is thus a widely recognized need for, and it would be highly advantageous to have, a method of targeting biofilms to thereby treat bacterial infections.

The formation of biofilms by the gram negative Escherichia coli bacterium is facilitated by the establishment of amyloid fibril networks [Chapman et al., (2002) Science 295:851-855]. These same networks are prevalent in a variety of diseases of unrelated origin where they may be found in various tissues and organs. Amongst these diseases are: Alzheimer's disease, Type II diabetes, Parkinson's disease, Prion diseases (such as the bovine spongiform encephalopathy—BSE) and various familial and systemic amyloidoses [Gazit (2002) Curr. Med. Chem. 9: 1725-1735].

More recent studies have revealed that amyloid structures play a role in the formation of aerial hyphae by the Gram-positive Streptomyces coelicolor bacteria [Elliot, M. A., et al., (2003), Genes Dev. 17, 1727-1740] and in the process of melanosome biogenesis in mammalian melanocytes [Berson, J. F. et al., (2003), J. Cell. Biol., 161, 521-533]. Amyloid formation was also suggested as being the underlying mechanism for prion formation in yeast [Tuite, M. F. & Cox, B. S. (2003), Nat. Rev. Mol. Cell. Biol. 4, 878-890; True, H. L., et al., (2004), Nature 431, 184-187]. It was hypothesized that the transition between prionic states should provide evolutionary advantage to the yeast. By allowing translational read-through events in [PSI⁺] yeast harbouring prions, a subset of the population could acquire new traits and endure fluctuating environments [True, H. L., et al., (2004), Nature 431, 184-187].

In all of these cases, monomeric proteins accumulate by a process of self-assembly, to form large deposits comprised of thousands of proteins that are associated into well-ordered structures. When examined by electron microscopy (EM) or atomic forces microscopy (AFM), the polypeptide deposits revealed typical fibrillar structures with a diameter of several nm and a length that can reach several microns. The well-ordered nature of the fibrils is evident by X-ray fiber diffraction which shows a clear 4.6-4.8 Å reflection on the meridian. Such reflection correlates with the hydrogen bonding distance between stacked β-strands. This is consistent with the predominantly β-sheet structure of the proteins present in amyloid deposits as determined by Fourier-transform infrared (FT-IR) and circular dichroism (CD) spectroscopy.

The EM analysis of the E. coli biofilms demonstrated the existence of a nanometeric network of fibrillar structures composed of the Curli protein [Chapman et al. (2002) Science 295:851-855]. Purified Curli protein was also shown to undergo a spontaneous transition from a random coil into a β-sheet rich structure, imitating the process which occurs in amyloid disease where soluble cellular proteins undergo a self-assembly process that leads to the formation of large and well-ordered protein deposits.

A similar analysis was performed on amyloid fibrils produced by the filamentous bacteria Streptomyces coelicolor. These bacteria produce aerial structures that facilitates the dispersion of infectious spores [Talbot N.J. (2003) Curr Biol. 13:R696-8]. Chaplins, a family of secreted, surface-active proteins, have been identified in Streptomyces coelicolor as being responsible for the formation of amyloid fibrils. As in the case of Curli fibrils, Chaplin fibrils can be visualized by EM, displaying typical β-sheet structures using circular dichroism. Therefore, Streptomyces provides another example of a bacterial system in which amyloid fibrils play a key role in the common non-planktonic state of bacteria.

Both curli [Chapman et al., (2002) Science 295:851-855] and chaplin fibrils [Claessen et al. (2003) Genes Dev. 17: 1714-1726] interact with thioflavin T (ThT). In addition, curli fibrils were shown to interact with Congo red (CR) [Chapman et al., (2002) Science 295:851-855]. Both of these dyes are used for the routine identification of amyloid fibrils formed by disease-related amyloidogenic polypeptides, such as the β-Amyloid (Aβ) polypeptide, which is the major constituent of fibrillar plaques formed in the case of Alzheimer's disease, and the islet amyloid polypeptide (IAPP) formed in the case of Type II diabetes.

While the mechanism of biofilm formation has been shown to involve an amyloid-like mechanism, to date the use of anti amyloid agents for treating bacterial infection has never been suggested nor shown.

### SUMMARY OF THE INVENTION

According to one aspect of the present invention there is provided a method of preventing or treating a pathogen infection in a subject, the method comprising administering to a subject in need thereof a therapeutically effective amount of an anti amyloid agent, thereby treating or preventing the pathogen infection in the subject.

According to another aspect of the present invention there is provided use of an anti-amyloid agent for the manufacture of a medicament identified for preventing or treating a pathogen infection in a subject.

According to yet another aspect of the present invention there is provided a method of typing a pathogen, the method comprising monitoring an alteration in growth and/or infectivity of the pathogen in the presence of an anti-amyloid agent, thereby typing the pathogen.

According to still another aspect of the present invention there is provided a method of identifying an anti-amyloid agent, the method comprising: (a) contacting molecules with an amyloid forming pathogen: and (b) identifying at least one molecule of the molecules capable of altering amyloid formation of the amyloid forming pathogen, thereby identifying the anti-amyloid agent.

According to an additional aspect of the present invention there is provided a medical device comprising an anti-amyloid agent attached thereto.

According to further features in preferred embodiments of the invention described below, the medical device is an intracorporeal device.

According to still further features in the described preferred embodiments the medical device is an extracorporeal device.

According to still further features in the described preferred embodiments the pathogen infection comprises a bacterial infection.

According to still further features in the described preferred embodiments the pathogen infection comprises a fungi infection.

According to still further features in the described preferred embodiments the anti-amyloid agent is a proteinaceous agent.

According to still further features in the described preferred embodiments the proteinaceous agent is a peptide agent.

According to still further features in the described preferred embodiments the peptide agent comprises an amino acid sequence selected from the group consisting of SEQ ID NO: 9, 10 and 11.

According to still further features in the described preferred embodiments the peptide agent comprises an amino acid sequence X-Y or Y-X, wherein X is an aromatic amino acid and Y is any amino acid other than glycine, the peptide being at least 2 and no more than 15 amino acids in length.

According to still further features in the described preferred embodiments at least one amino acid of the amino acid sequence of the peptide is a D stereoisomer.

According to still further features in the described preferred embodiments at least one amino acid of the amino acid sequence of the peptide is an L stereoisomer.

According to still further features in the described preferred embodiments Y is a polar uncharged amino acid selected from the group consisting of serine, threonine, asparagine, glutamine and natural derivatives thereof.

According to still further features in the described preferred embodiments Y is a β-sheet breaker amino acid.

According to still further features in the described preferred embodiments the β-sheet breaker amino acid is a naturally occurring amino acid.

According to still further features in the described preferred embodiments the naturally occurring amino acid is selected from the group consisting of proline, aspartic acid, glutamic acid, glycine, lysine and serine.

According to still further features in the described preferred embodiments the β-sheet breaker amino acid is a synthetic amino acid.

According to still further features in the described preferred embodiments the synthetic amino acid is a Cα-methylated amino acid.

According to still further features in the described preferred embodiments the Cα-methylated amino acid is α-aminoisobutyric acid.

According to still further features in the described preferred embodiments the peptide is a linear or cyclic peptide.

According to still further features in the described preferred embodiments the peptide is two amino acids in length and Y is a β-sheet breaker amino acid.

According to still further features in the described preferred embodiments the peptide is 3 amino acids in length, whereas Y is an aromatic amino acid and an amino acid residue attached to the amino acid sequence X-Y or Y-X is a β-sheet breaker amino acid.

According to still further features in the described preferred embodiments the β-sheet breaker amino acid is at a C-terminus of the peptide.

According to still further features in the described preferred embodiments the peptide is at least 4 amino acids in length and includes at least two serine residues at a C-terminus thereof.

According to still further features in the described preferred embodiments the peptide is at least 3 amino acids in length and includes a thiolated amino acid at an N-terminus thereof.

According to still further features in the described preferred embodiments the peptide is at least 3 amino acids in length and whereas at least one of the amino acids of the peptide other than X-Y is a β-sheet breaker amino acid.

According to still further features in the described preferred embodiments the peptide is at least 3 amino acids in length and whereas at least one of the amino acids of the peptide is a positively charged amino acid and at least one of the amino acids of the peptide is a negatively charged amino acid.

According to still further features in the described preferred embodiments the proteinaceous agent is an antibody.

According to still further features in the described preferred embodiments the agent is a non-proteinaceous agent.

According to still further features in the described preferred embodiments the non-proteinaceous agent comprises a compound having the general Formula I:

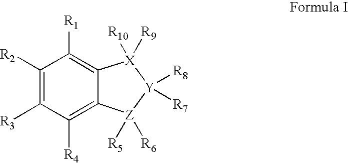

a pharmaceutically acceptable salt thereof or a prodrug thereof,

wherein:

X, Y and Z are each independently selected from the group consisting of carbon, oxygen, sulfur, CR₁₁R₁₂ or R₁₃R₁₄C—CR₁₅R₁₆, provided that at least one of X, Y and Z is oxygen or sulfur; and

R₁-R₁₆ are each independently selected from the group consisting of hydrogen, lone pair electrons, hydroxy, alkyl, cycloalkyl, phenyl, alkoxyphenyl, thioalkoxyphenyl, aryloxyphenyl, thioaryloxyphenyl, carboxyphenyl, thiocarboxyphenyl, phenol, hydroxyphenol, dihydroxyphenol, aryl, alkenyl, alkynyl, heteroaryl, heteroalicyclic, halo, alkoxy, aryloxy, thiohydroxy, thioalkoxy, thioaryloxy, C-carboxy, O-carboxy, thiocarboxy, carbonyl, oxo, thiocarbonyl, sulfinyl, and sulfonyl, or absent, or, alternatively, at least two of R₁-R₄ and/or at least two of R₅-R₁₆ form at least one five- or six-membered aromatic, heteroaromatic, alicyclic or heteroalicyclic ring,

whereas:

at least one of R₁-R₄ is selected from the group consisting of hydroxy, thiohydroxy, alkoxy, thioalkoxy, aryloxy, thioaryloxy, carboxy and thiocarboxy; and/or

at least one of R₅-R₁₆ comprises phenol, alkoxyphenyl, thioalkoxyphenyl, aryloxyphenyl, thioaryloxyphenyl, carboxyphenyl, thiocarboxyphenyl, hydroxyphenol, and dihydroxyphenol,

with the proviso that the compound is not any one of catechin, epicatechin, gallocatechin gallate, epigallocatechin gallate, epigallocatechin, and epicatechin gallate,

for the manufacture of a medicament identified for the treatment of amyloid-associated diseases.

According to still further features in the described preferred embodiments,

X is carbon;

Y is R₁₃R₁₄C—CR₁₅R₁₆; and

Z is oxygen.

According to still further features in the described preferred embodiments,

R₉ is oxo; and

R₁₀ is absent.

According to still further features in the described preferred embodiments at least one of R₁₃-R₁₆ is selected from the group consisting of alkoxyphenyl, thioalkoxyphenyl, aryloxyphenyl, thioaryloxyphenyl, carboxyphenyl, thiocarboxyphenyl, phenol, hydroxyphenol and dihydroxyphenol.

According to still further features in the described preferred embodiments each of R₁ and R₃ is hydroxy.

According to still further features in the described preferred embodiments at least one of R₁₃-R₁₆ is alkyl.

According to still further features in the described preferred embodiments,

X is carbon;

Y is oxygen;

Z is carbon or sulfur; and

at least one of R₅ and R₆ is oxo.

According to still further features in the described preferred embodiments R₉ and R₁₀ is selected from the group consisting of alkoxyphenyl, thioalkoxyphenyl, aryloxyphenyl, thioaryloxyphenyl, carboxyphenyl, thiocarboxyphenyl, phenol, hydroxyphenol and dihydroxyphenol.

According to still further features in the described preferred embodiments the compound is selected from the group consisting of phenol red, dimethoxy phenol red, methoxy phenol red, diacetoxy phenol red, acetoxy phenol red, pyrocatechol violet, phenolphthaleine, catechin, epigallocatechin gallate, epicatechin gallate, epicatechin, epigallocatechin, eriodictyol, quercetin, procyanidin, hydroxyphenyl, tocopherol, and bromophenol red.

According to still further features in the described preferred embodiments the non peptide agent comprises a compound having the general formula:

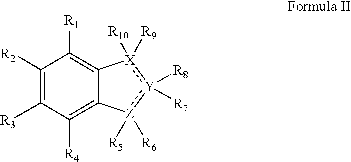

a pharmaceutically acceptable salt thereof, or a prodrug thereof,
wherein:

the dashed line denotes a double bond either between X and Y, or, between Y and Z;

X, Y and Z are each independently selected from the group consisting of carbon and nitrogen, whereas at least one of X, Y, and Z is nitrogen; and

R₁-R₁₀ are each independently selected from the group consisting of hydrogen, lone pair electrons, hydroxy, alkyl, cycloalkyl, phenyl, phenol, hydroxyphenol, dihydroxyphenol, aryl, alkenyl, alkynyl, heteroaryl, heteroalicyclic, halo, alkoxy, aryloxy, thiohydroxy, thioalkoxy, thioaryloxy, C-carboxy, O-carboxy, thiocarboxy, carbonyl, oxo, thiocarbonyl, sulfinyl, and sulfonyl, or absent, or, alternatively, at least two of R₁-R₁₀ form at least one five- or six-membered aromatic, heteroaromatic, alicyclic or heteroalicyclic ring,

thereby treating the amyloid associated disease in the subject.

According to still further features in the described preferred embodiments,

each of X and Y is carbon; and

Z is nitrogen.

According to still further features in the described preferred embodiments the double bond is between X and Y.

According to still further features in the described preferred embodiments at least one of R₁-R₁₀ comprises a hydroxy group.

According to still further features in the described preferred embodiments at least one of R₁-R₁₀ comprises a hydroxy group.

According to still further features in the described preferred embodiments at least one of R₁ and R₉ comprises a hydroxy group.

According to still further features in the described preferred embodiments at least one of R₁ and R₉ is a hydroxy group.

According to still further features in the described preferred embodiments each of R₂-R₅ and R₇ is hydrogen and R₆, R₈ and R₁₀ are absent.

According to still further features in the described preferred embodiments R₁ is hydrogen and R₉ is a hydroxy group.

According to still further features in the described preferred embodiments R₁ is a hydroxy group and R₉ is hydrogen.

According to still further features in the described preferred embodiments at least one of R₁-R₁₀ is a hydroxyalkyl.

According to still further features in the described preferred embodiments at least one of R₇ and R₉ is a hydroxyalkyl.

According to still further features in the described preferred embodiments each of R₁-R₅ is hydrogen and R₆, R₈ and R₁₀ are absent.

According to still further features in the described preferred embodiments the hydroxyalkyl is hydroxymethyl.

According to still further features in the described preferred embodiments R₇ is hydrogen and R₉ is the hydroxymethyl.

According to still further features in the described preferred embodiments each of R₁-R₅ is hydrogen and R₆, R₈ and R₁₀ are absent.

According to still further features in the described preferred embodiments each of R₇ and R₉ is a hydroxyalkyl.

According to still further features in the described preferred embodiments the non-proteinaceous agent is a non-steroidal anti-inflammatory drug.

According to still further features in the described preferred embodiments the non-proteinaceous agent is selected from the group consisting of nicotine, acridine, acridine orange, methylene blue, congo red, thioflavin-T and tetracycline.

The present invention successfully addresses the shortcomings of the presently known configurations by providing methods of treating and detecting pathogen infection using anti-amyloid agents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-c depict structural and functional similarities between prion proteins and CsgA protein of bacteria. FIG. 1a-Shows sequence analysis of CsgA and AgfA proteins which reveals short peptide repeats that are reminiscent the PrP and Sup35 repeats, both in length (6-9 residues) and in chemical composition. Aligned oligopeptide repeats are indicated in grey background. Highly conserved residues within the repeated domains are indicated as bold letters and colored according to residue type in red (aromatic), black (glycine) or blue (glutamine/aspargine). FIG. 1b shows TEM analysis of the self-assembly properties of oligopeptide repeats. The QFGGGN (SEQ ID NO: 3) and QHGGGN (SEQ ID NO: 5) peptides assembled into fibrillar structures. This ability was lost with the substitution of the second residue to alanine (QAGGGN, SEQ ID NO: 4). FIG. 1c shows TEM analysis of scrambled QHGGGN (SEQ ID NO: 5) peptide sequences.

FIGS. 2a-b are graphs showing copper binding to QHGGGN (SEQ ID NO: 5) peptide repeat. FIG. 2a shows Circular Dichroism (CD) spectra, 250-800 nm, of the curli QHGGGN (SEQ ID NO: 5) hexapeptide repeat in the absence (open circles) and presence (closed circles) of equimolar ratio of CuCl₂. FIG. 2b shows Cu²⁺ binding curve for the QHGGGN (SEQ ID NO: 5) peptide. Changes in CD signals with increasing amounts of Cu²⁺ were measured at 590 nm, indicating equimolar binding stoichiometry. The titration of metal ions to the oligopeptide was performed using small aliquots from stock aqueous solutions of 25 mM CuCl₂.2H₂O. CD spectrum (250-800 nm) of QHGGGN (0.5 mM, pH 7.5) was measured with the addition of Cu²⁺ in increments of 0.0125 mM (as a minimum) of CuCl₂ (0.5 μl) from 0 up to 0.65 mM. Typically, 50 mM HEPES pH 7.5 buffer was used for CD studies. Spectra were obtained using an AVIV spectrapolarimeter and a 5 mm (FIG. 2a) and 10 mm (FIG. 2b) path length cuvettes.

FIG. 3a shows the effect of anti amyloid peptides on amyloid formation as determined by quantitative Congo-red binding assay. Binding units represent the decrease in absorbance (A₄₈₇) of the CR solution after incubation with bacteria.

FIG. 3b shows the effect of anti amyloid peptides of the present invention on Fibronectin binding. The adhesion of bacteria to fibronectin coated wells was determined by measuring absorbance at 405 nm. For (A) and (B), each column is the average of three experiments. Error bars represent standard errors; asterisks represent p<0.05 (compared to XL1-Blue(pMRInv) values), as determined by a paired sample t-test.

FIG. 3c shows Electron-microscopy micrographs of curli expressing bacteria, grown to early stationary phase in the presence or absence of QFGGGNPP (SEQ ID NO: 11) peptide. E. coli K-12 XL1-Blue(pMRInv) were grown until A₆₀₀, 1.1 with and without 0.3 mM of the inhibitor peptide QFGGGNPP (SEQ ID NO: 11). 400-mesh copper grids were coated with 5 μl of the suspension bacteria and allowed to sediment for 1 min on a grid. Scale bars represent 1 μm.

FIGS. 4a-c show the effect of anti-amyloid peptide agent on the internalization of biofilm producing bacteria into eukaryotic host cells, as determined by an Internalization assay. FIG. 4a-Representative results of bacterial colony forming units (cfu) following adhesion and internalization to Human Embryo Kidney 293 cells (HEK293). E. coli strain XL1-Blue(pMRInv) and E. coli strain XL1-Blue (upper and lower windows, respectively) were quantified for internalization in the absence or presence (left and right windows, respectively) of 0.4 mM peptide inhibitor by a standard antibiotic protection assay. FIG. 4b shows mean results of two independent internalization assays composed of eight internal repeats. Error bars represent standard error, where asterisk represent p<0.01 as determined by a paired sample t-test. FIG. 4c shows mean results of two independent internalization assays composed of four internal repeats, using 0.4 mM QFGGGN peptide (SEQ ID NO: 3) instead of the QFGGGNPP (SEQ ID NO: 11). Error bars represent standard error.

### DESCRIPTION OF THE PREFERRED EMBODIMENTS

The present invention is of a novel use of anti-amyloid agents for treating pathogenic infections. Specifically, the present invention can be used to treat or prevent

The principles and operation the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Amyloid fibrils have historically been associated with pathology in a class of degenerative diseases including Alzheimer's disease, diabetes and Creutzfeld-Jacob disease. However, recent data have shown that amyloid fibril formation not only results in toxic aggregates but also provides biologically functional molecules [Kelly (2003) J. Cell. Biol. 161:461-2]. Such functional amyloids have been identified on the surfaces of fungi and bacteria and suggested to be involved in the process of biofilm formation.

While reducing the present invention to practice, the present inventors uncovered that anti-amyloid agents can be used for treating and/or preventing pathogenic infections, probably by inhibiting formation or disintegrating a pre-existing biofilm.

As is illustrated in the Examples section which follows, the present inventors were able to show functional and structural similarity between oligopeptide repeats of the major curlin protein (i.e., amyloid forming protein of bacteria) and those of animal and yeast prions (see Example 1 of the Examples section which follows). Synthetic peptides generated according to these oligopeptide repeats were able to self-assemble and form fibrillar structures (see Examples 2-3 of the Examples section which follows). Furthermore, conjugation of β-breaker elements to the prion-like repeat (Example 4 of the Examples section which follows) significantly inhibited amyloid formation and cell invasion of curli expressing bacteria, supporting therapeutic use of these peptides, and anti-amyloid agents in general, for treating or preventing infections elicited by amyloid forming microorganisms (Example 5 of the Examples section, which follows).

Thus according to one aspect of the present invention there is provided a method of preventing or treating a pathogen infection in a subject. The method according to this aspect of the present invention is effected by administering to a subject in need thereof, a therapeutically effective amount of an anti amyloid agent, thereby treating or preventing the pathogen infection in the subject.

As used herein the phrase “subject in need thereof” refers to an organism (e.g., a warm blooded organism) infectable (i.e., infected or being at risk of infection) with the pathogen of the present invention. Preferably, the subject according to this aspect of the present invention is a mammalian subject, preferably a human subject.

As used herein the term “pathogen” refers to an amyloid forming microorganism capable of causing a disease in the infected subject. Examples of such microorganisms include, but are not limited to, bacteria and fungi.

Examples of amyloid forming bacteria include both Gram positive bacteria [e.g., streptomycetes, wherein amyloid formation is attributed to the CHAPLIN proteins; see e.g., Claessen (2003) Genes Dev. 17:1714-1726; Claessen (2004) Mol. Microbiol. 53:433-443; Elliot (2003) Genes Dev. 17:1727-1740] and Gram negative bacteria [e.g., Escherichia and Salmonella Sp. wherein amyloid formation is attributed to the CURLI and TAFI (also termed SEF17) proteins; see e.g., Chapman (2002) Science 295:851-855].

Examples of amyloid forming fungi include ascomycetes and basidiomycetes phyla, where Hydrophorbins are implicated in amyloid formation [Wosten (1993) Plant Cell 5:1567-1574] as well as yeast.

As used herein the term “amyloid” refers to fibrillar amyloid as well as aggregated but not fibrillar amyloid, hereinafter “protofibrillar amyloid”, which may be pathogenic as well.

As used herein the phrase “anti amyloid agent” refers to an agent which is capable of inhibiting amyloid aggregate formation or disrupting pre-assembled amyloid aggregates [see e.g., Gazit, E. (2002) Curr. Med. Chem. 9: 1725-1735; Sacchettini (2002) Nat Rev Drug Discov 1:267-275].

The anti amyloid agent of the present invention may be any protein anti-amyloid agent or a non-protein anti-amyloid agent which is known in the art.

The following provides examples of protein and non-protein anti-amyloid agents which can be used in accordance with the present invention.

Proteinaceous Agents

Peptide Agents

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)—CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, Tic, naphtylalanine (NaI), phenylisoserine, threoninol, ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1) and non-conventional or modified amino acids (e.g., synthetic, Table 2) which can be used with the present invention.

TABLE 1

| | Three-Letter | |
|---|---|---|
| Amino Acid | Abbreviation | One-letter Symbol |
| alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| glycine | Gly | G |
| Histidine | His | H |
| isoleucine | Iie | I |
| leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane-carboxylate | Cpro | L-N-methylasparagine | Nmasn |
| | | L-N-methylaspartic acid | Nmasp |
| aminoisobutyric acid | Aib | L-N-methylcysteine | Nmcys |
| aminonorbornyl-carboxylate | Norb | L-N-methylglutamine | Nmgin |
| | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenylethylamino)cyclopropane | Nmbc | | |

The peptides of the present invention are preferably utilized in a linear form, although it will be appreciated that in cases where cyclization does not severely interfere with peptide characteristics, cyclic forms of the peptide can also be utilized.

Cyclic peptides can either be synthesized in a cyclic form or configured so as to assume a cyclic form under desired conditions (e.g., physiological conditions).

For example, a peptide according to the teachings of the present invention can include at least two cysteine residues flanking the core peptide sequence. In this case, cyclization can be generated via formation of S—S bonds between the two Cys residues. Side-chain to side chain cyclization can also be generated via formation of an interaction bond of the formula —(—CH2-)n-S—CH2-C—, wherein n=1 or 2, which is possible, for example, through incorporation of Cys or homoCys and reaction of its free SH group with, e.g., bromoacetylated Lys, Orn, Dab or Dap. Furthermore, cyclization can be obtained, for example, through amide bond formation, e.g., by incorporating Glu, Asp, Lys, Orn, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH2)n-COOH)—C(R)H—COOH or H—N((CH2)n-COOH)—C(R)H—NH2, wherein n=1-4, and further wherein R is any natural or non-natural side chain of an amino acid.

Thus, a peptide agent of the present invention may comprise the amino acid sequence X-Y or Y-X, wherein X is an aromatic amino acid and Y is any amino acid. As is shown in WO05000193 to the present inventor, the present inventor have uncovered that contrary to the teachings of the prior art, it is aromaticity rather than hydrophobicity, which dictates amyloid self-assembly. Thus, the aromatic amino acid of the peptides of the present invention is pivotal to the formation of amyloid fibrils.

According to an embodiment of the present invention Y is any amino acid other than glycine.

The aromatic amino acid can be any naturally occurring or synthetic aromatic residue including, but not limited to, phenylalanine, tyrosine, tryptophan, phenylglycine, or modificants, precursors or functional aromatic portions thereof. Examples of aromatic residues which can form a part of the peptides of present invention are provided in Table 2 above.

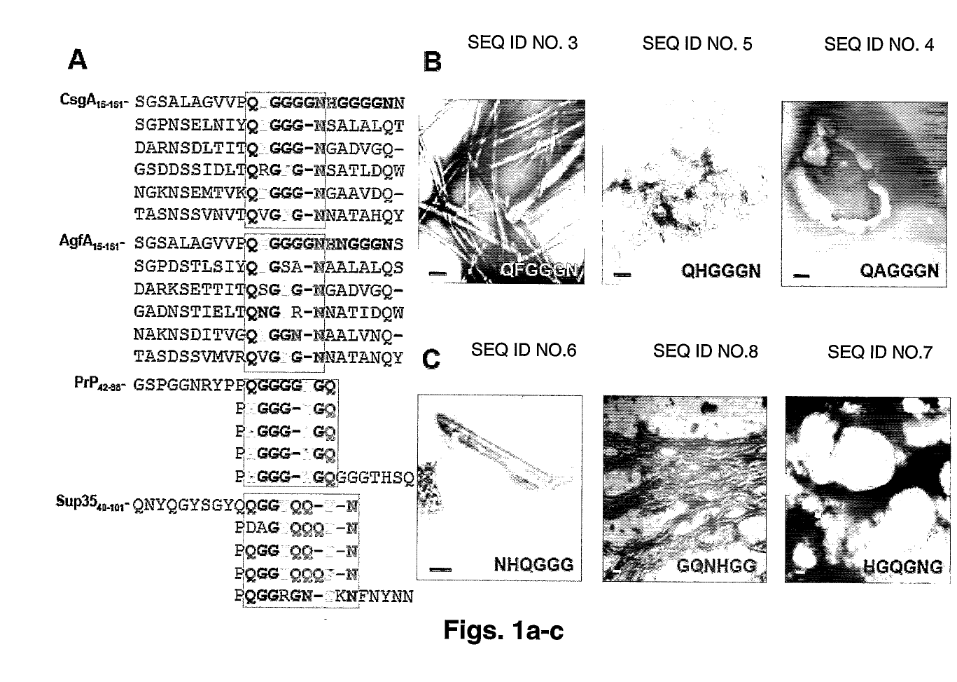

Since aggregation kinetics and aggregate structures are largely determined by the specific residue composition and possibly the length of the peptides generated (see FIG. 1), the present invention encompasses both longer peptides (e.g., 10-50 amino acids) or preferably shorter peptides (e.g., 2-15 amino acids, preferably at least 2, at least 3, at least 4, at least 5, at least 6, at least 8, at least 10, say 12 amino acids, preferably no more than 15 amino acids) including any of these sequences (See SEQ ID NOs. 1-11).

In order to enhance the rate of amyloid formation, the peptides of the present invention preferably include at least one polar and uncharged amino acid including but not limited to serine, threonine, asparagine, glutamine or natural or synthetic derivatives thereof (see Table 2).

According to one embodiment of this aspect of the present invention, amino acid residue Y is the polar and uncharged amino acid.

According to another embodiment of this aspect of the present invention, the peptide includes at least 3 amino acids, the X-Y/Y-X amino acid sequence described hereinabove and an additional polar and uncharged amino acid positioned either upstream (N-Terminal end) or downstream (C-Terminal end) of the X-Y/Y-X sequence.

The peptides of the present invention, can be at least 3 amino acid in length and may include at least one pair of positively charged (e.g., lysine and arginine) and negatively charged (e.g., aspartic acid and glutamic acid) amino acids.

Yet additionally, the peptide of the present invention can be 4 amino acids in length and include two serine residues at the C-terminal end of the X-Y/Y-X sequence.

The peptides of the present invention preferably include at least one β-sheet breaker amino acid residue, which is positioned in the peptide sequence as described below. Peptides which include such α-sheet breaker amino acids retain recognition of amyloid polypeptides but prevent aggregation thereof (see WO05000193). According to one preferred embodiment of this aspect of the present invention, the β-sheet breaker amino acid is a naturally occurring amino acid such as proline (e.g., SEQ ID NOs. 9-11) which is characterized by a limited phi angle of about −60 to +25 rather than the typical beta sheet phi angle of about −120 to −140 degrees, thereby disrupting the beta sheet structure of the amyloid fibril. Other β-sheet breaker amino acid residues include, but are not limited to aspartic acid, glutamic acid, glycine, lysine and serine (according to Chou and Fasman (1978) Annu. Rev. Biochem. 47, 258).

According to another preferred embodiment of this aspect of the present invention, the β-sheet breaker amino acid residue is a synthetic amino acid such as a Cα-methylated amino acid, which conformational constrains are restricted [Balaram, (1999) J. Pept. Res. 54, 195-199]. Unlike natural amino acids, Cα-methylated amino acids have a hydrogen atom attached to the C_{α}, which affects widely their sterical properties regarding the φ and ψ angels of the amide bond. Thus, while alanine has a wide range of allowed φ and ψ conformations, α-aminoisobutyric acid (Aib, see Table 2, above) has limited φ and ψ conformations. Hence, peptides of the present invention which are substituted with at least one Aib residue are capable of binding amyloid polypeptides but prevent aggregation thereof.

The β-sheet breaker amino acid of this aspect of the present invention can be located at position Y of the X-Y/Y-X amino acid sequence of the peptide. Alternatively, the peptides of this aspect of the present invention can be at least 3 amino acids and include the breaker amino acid in any position other than the X-Y/Y-X amino acid sequence.

The β-sheet breaker amino acid may be positioned upstream of the aromatic residue or downstream thereto (see SEQ ID NO: 11) or both upstream and down stream to the aromatic residue (SEQ ID NOs. 9-10).

According to one preferred embodiment of this aspect of the present invention the peptide is three amino acids in length, wherein Y is an aromatic amino acid and an amino acid residue attached to the amino acid sequence X-Y or Y-X is a β-sheet breaker amino acid, which is preferably attached at the C-terminus of the peptide.

According to another preferred embodiment of this aspect of the present invention the peptide is two amino acids in length and Y is a β-sheet breaker amino acid.

Since the present peptide agents of the present invention are utilized in therapeutics which requires the peptides to be in soluble form, the peptides of the present invention preferably include one or more non-natural or natural polar amino acids, including but not limited to serine and threonine which are capable of increasing peptide solubility due to their hydroxyl-containing side chain.

According to a preferred embodiment of the present invention the peptide is a dipeptide having the following general formula:

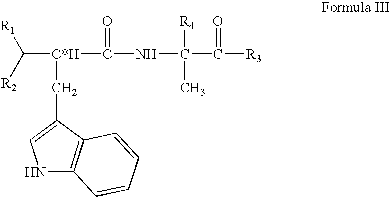

wherein:
C* is a chiral carbon having a D configuration (also referred to in the art as R-configuration).

R₁ and R₂ are each independently selected from the group consisting of hydrogen, alkyl, cycloalkyl, aryl, carboxy, thiocarboxy, C-carboxylate and C-thiocarboxylate;

R₃ is selected from the group consisting of hydroxy, alkoxy, aryloxy, thiohydroxy, thioalkoxy, thioaryloxy, halo and amine; and

R₄ is alkyl.

As used herein, the term “alkyl” refers to a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. When substituted, the substituent group can be, for example, halo, hydroxy, cyano, nitro and amino.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, halo, hydroxy, cyano, nitro and amino.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, alkyl, cycloalkyl, halo, hydroxy, alkoxy, thiohydroxy, thioalkoxy, cyano, nitro and amino.

A “hydroxy” group refers to an —OH group.

An “alkoxy” group refers to both an —O-alkyl and an —O-cycloalkyl group, as defined herein.

An “aryloxy” group refers to an —O-aryl group, as defined herein.

A “thiohydroxy” group refers to a —SH group.

A “thioalkoxy” group refers to both an —S-alkyl group, and an —S-cycloalkyl group, as defined herein.

A “thioaryloxy” group refers to an —S-aryl group, as defined herein.

A “carboxy” group refers to a —C(═O)—R′ group, where R′ is hydrogen, halo, alkyl, cycloalkyl or aryl, as defined herein.

A “thiocarboxy” group refers to a —C(═S)—R′ group, where R′ is as defined herein for R′.

A “C-carboxylate” group refers to a —C(═O)—O—R′ groups, where R′ is as defined herein.

A “C-thiocarboxylate” group refers to a —C(═S)—O—R′ groups, where R′ is as defined herein.

A “halo” group refers to fluorine, chlorine, bromine or iodine.

An “amine” group refers to an —NR′R″ group where R′ is as defined herein and R″ is as defined for R′.

A “nitro” group refers to an —NO₂ group.

A “cyano” group refers to a —C≡N group.

Preferably, R₄ is methyl, such that the compound above is D-tryptophane-alpha-aminobutyric acid (also referred to herein as D-Trp-aib or D-tryptophane-alpha-methyl-alanine), or a derivative thereof.

It will be appreciated that unmodified di-peptides, peptides of L-configuration, peptides which are of a reversed configuration (i.e., C-to-N sequence of tryptophane (D/L) and alpha-methyl alanine), or alternatively, macromolecules (e.g., peptides, immobilized peptides) which encompass the above-described peptide sequence, are known (see e.g., WO 02/094857, WO 02/094857, EP Pat. No. 966,975, U.S. Pat. Nos. 6,255,286, 6,251,625, 6,162,828 and 5,304,470). However, such molecules are chemically and biologically different than the above described peptide, which unique activity is strictly dependent on its structure.

The peptides of the present invention may be synthesized by any techniques that are known to those skilled in the art of peptide synthesis. For solid phase peptide synthesis, a summary of the many techniques may be found in: Stewart, J. M. and Young, J. D. (1963), “Solid Phase Peptide Synthesis,” W. H. Freeman Co. (San Francisco); and Meienhofer, J (1973). “Hormonal Proteins and Peptides,” vol. 2, p. 46, Academic Press (New York). For a review of classical solution synthesis, see Schroder, G. and Lupke, K. (1965). The Peptides, vol. 1, Academic Press (New York).

In general, peptide synthesis methods comprise the sequential addition of one or more amino acids or suitably protected amino acids to a growing peptide chain. Normally, either the amino or the carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth; traditionally this process is accompanied by wash steps as well. After all of the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide, and so forth. Further description of peptide synthesis is disclosed in U.S. Pat. No. 6,472,505. A preferred method of preparing the peptide compounds of the present invention involves solid-phase peptide synthesis, utilizing a solid support. Large-scale peptide synthesis is described

It will be appreciated that peptide agents of the present invention may also be synthesized by recombinant DNA techniques and provided to the subject as such. Alternatively, the peptides of the present invention may be provided to the subject by ex vivo or in vivo gene therapy techniques. Such recombinant techniques are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

Antibody Agents

Antibody agents of the present invention are capable of specifically binding the amyloid forming unit of the amyloid forming polypeptide (e.g., chaplin, curli, tafi and the like), thereby inhibiting amyloid formation and even disintegrating pre-assembled aggregates (dependent on the antibody affinity). Thus for example, an antibody agent of the present invention may be directed at the QFGGGN amyloid forming unit of curli (as described in Example 1 of the Examples section which follows).

As used herein, the term “antibody” refers to a substantially intact antibody molecule or an antibody fragment.

As used herein, the phrase “antibody fragment” refers to a functional fragment of an antibody that is capable of binding to an antigen.

Suitable antibody fragments for practicing the present invention include, inter alia, a complementarity-determining region (CDR) of an immunoglobulin light chain (referred to herein as “light chain”), a CDR of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single-chain Fv, an Fab, an Fab′, and an F(ab′)2.

Functional antibody fragments comprising whole or essentially whole variable regions of both light and heavy chains are defined as follows:

(i) Fv, defined as a genetically engineered fragment consisting of the variable region of the light chain and the variable region of the heavy chain expressed as two chains;

(ii) single-chain Fv (“scFv”), a genetically engineered single-chain molecule including the variable region of the light chain and the variable region of the heavy chain, linked by a suitable polypeptide linker.

(iii) Fab, a fragment of an antibody molecule containing a monovalent antigen-binding portion of an antibody molecule, obtained by treating whole antibody with the enzyme papain to yield the intact light chain and the Fd fragment of the heavy chain, which consists of the variable and CH1 domains thereof;

(iv) Fab′, a fragment of an antibody molecule containing a monovalent antigen-binding portion of an antibody molecule, obtained by treating whole antibody with the enzyme pepsin, followed by reduction (two Fab′ fragments are obtained per antibody molecule); and

(v) F(ab′)2, a fragment of an antibody molecule containing a monovalent antigen-binding portion of an antibody molecule, obtained by treating whole antibody with the enzyme pepsin (i.e., a dimer of Fab′ fragments held together by two disulfide bonds).

Methods of generating monoclonal and polyclonal antibodies are well known in the art. Antibodies may be generated via any one of several known methods, which may employ induction of in vivo production of antibody molecules, screening of immunoglobulin libraries (Orlandi, R. et al. (1989). Cloning immunoglobulin variable domains for expression by the polymerase chain reaction. Proc Natl Acad Sci USA 86, 3833-3837; and Winter, G. and Milstein, C. (1991). Man-made antibodies. Nature 349, 293-299), or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler, G. and Milstein, C. (1975). Continuous cultures of fused cells secreting antibody of predefined specificity. Nature 256, 495-497; Kozbor, D. et al. (1985). Specific immunoglobulin production and enhanced tumorigenicity following ascites growth of human hybridomas. J. Immunol. Methods 81, 31-42; Cote R J. et al. (1983). Generation of human monoclonal antibodies reactive with cellular antigens. Proc Natl Acad Sci USA 80, 2026-2030; and Cole, S. P. et al. (1984). Human monoclonal antibodies. Mol Cell Biol 62, 109-120).

In cases where target antigens are too small to elicit an adequate immunogenic response when generating antibodies in vivo, such antigens (referred to as “haptens”) can be coupled to antigenically neutral carriers such as keyhole limpet hemocyanin (KLH) or serum albumin (e.g., bovine serum albumin (BSA)) carriers (see, for example, U.S. Pat. Nos. 5,189,178 and 5,239,078). Coupling a hapten to a carrier can be effected using methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill., USA. The resulting immunogenic complex can then be injected into suitable mammalian subjects such as mice, rabbits, and others. Suitable protocols involve repeated injection of the immunogen in the presence of adjuvants according to a schedule designed to boost production of antibodies in the serum. The titers of the immune serum can readily be measured using immunoassay procedures which are well known in the art.

The antisera obtained can be used directly or monoclonal antibodies may be obtained, as described hereinabove.

Antibody fragments may be obtained using methods well known in the art. (See, for example, Harlow, E. and Lane, D. (1988). Antibodies: A Laboratory Manual, Cold Spring Harbor Laboratory, New York.) For example, antibody fragments according to the present invention can be prepared by proteolytic hydrolysis of the antibody or by expression in E. Coli or mammalian cells (e.g., Chinese hamster ovary (CHO) cell culture or other protein expression systems) of DNA encoding the fragment.

Alternatively, antibody fragments can be obtained by pepsin or papain digestion of whole antibodies by conventional methods. As described hereinabove, an (Fab′)2 antibody fragments can be produced by enzymatic cleavage of antibodies with pepsin to provide a 5S fragment. This fragment can be further cleaved using a thiol reducing agent, and optionally a blocking group for the sulfhydryl groups resulting from cleavage of disulfide linkages, to produce 3.5S Fab′ monovalent fragments. Alternatively, enzymatic cleavage using pepsin produces two monovalent Fab′ fragments and an Fc fragment directly. Ample guidance for practicing such methods is provided in the literature of the art (for example, refer to: U.S. Pat. Nos. 4,036,945 and 4,331,647; and Porter, R. R. (1959). The hydrolysis of rabbit y-globulin and antibodies with crystalline papain. Biochem J 73, 119-126). Other methods of cleaving antibodies, such as separation of heavy chains to form monovalent light-heavy chain fragments, further cleavage of fragments, or other enzymatic, chemical, or genetic techniques may also be used, so long as the fragments retain the ability to bind to the antigen that is recognized by the intact antibody.

As described hereinabove, an Fv is composed of paired heavy chain variable and light chain variable domains. This association may be noncovalent (see, for example, Inbar, D. et al. (1972). Localization of antibody-combining sites within the variable portions of heavy and light chains. Proc Natl Acad Sci USA 69, 2659-2662). Alternatively, as described hereinabove, the variable domains may be linked to generate a single-chain Fv by an intermolecular disulfide bond, or alternately such chains may be cross-linked by chemicals such as glutaraldehyde.

Preferably, the Fv is a single-chain Fv. Single-chain Fvs are prepared by constructing a structural gene comprising DNA sequences encoding the heavy chain variable and light chain variable domains connected by an oligonucleotide encoding a peptide linker. The structural gene is inserted into an expression vector, which is subsequently introduced into a host cell such as E. coli. The recombinant host cells synthesize a single polypeptide chain with a linker peptide bridging the two variable domains. Ample guidance for producing single-chain Fvs is provided in the literature of the art (see, e.g.: Whitlow, M. and Filpula, D. (1991). Single-chain Fv proteins and their fusion proteins. METHODS: A Companion to Methods in Enzymology 2(2), 97-105; Bird, R. E. et al. (1988). Single-chain antigen-binding proteins. Science 242, 423-426; Pack, P. et al. (1993). Improved bivalent miniantibodies, with identical avidity as whole antibodies, produced by high cell density fermentation of Escherichia coli. Biotechnology (N.Y.) 11(11), 1271-1277; and U.S. Pat. No. 4,946,778).

Isolated complementarity-determining region peptides can be obtained by constructing genes encoding the CDR of an antibody of interest. Such genes may be prepared, for example, by RT-PCR of the mRNA of an antibody-producing cell. Ample guidance for practicing such methods is provided in the literature of the art (e.g., Larrick, J. W. and Fry, K. E. (1991). PCR Amplification of Antibody Genes. METHODS: A Companion to Methods in Enzymology 2(2), 106-110).

It will be appreciated that for human therapy or diagnostics, humanized antibodies are preferably used. Humanized forms of non-human (e.g., murine) antibodies are genetically engineered chimeric antibodies or antibody fragments having (preferably minimal) portions derived from non-human antibodies. Humanized antibodies include antibodies in which the CDRs of a human antibody (recipient antibody) are replaced by residues from a CDR of a non-human species (donor antibody), such as mouse, rat, or rabbit, having the desired functionality. In some instances, the Fv framework residues of the human antibody are replaced by corresponding non-human residues. Humanized antibodies may also comprise residues found neither in the recipient antibody nor in the imported CDR or framework sequences. In general, the humanized antibody will comprise substantially all of at least one, and typically two, variable domains, in which all or substantially all of the CDRs correspond to those of a non-human antibody and all or substantially all of the framework regions correspond to those of a relevant human consensus sequence. Humanized antibodies optimally also include at least a portion of an antibody constant region, such as an Fc region, typically derived from a human antibody (see, for example: Jones, P. T. et al. (1986). Replacing the complementarity-determining regions in a human antibody with those from a mouse. Nature 321, 522-525; Riechmann, L. et al. (1988). Reshaping human antibodies for therapy. Nature 332, 323-327; Presta, L. G. (1992b). Curr Opin Struct Biol 2, 593-596; and Presta, L. G. (1992a). Antibody engineering. Curr Opin Biotechnol 3(4), 394-398).

Methods for humanizing non-human antibodies are well known in the art. Generally, a humanized antibody has one or more amino acid residues introduced into it from a source which is non-human. These non-human amino acid residues are often referred to as imported residues, which are typically taken from an imported variable domain. Humanization can be performed essentially as described (see, for example: Jones et al. (1986); Riechmann et al. (1988); Verhoeyen, M. et al. (1988). Reshaping human antibodies: grafting an antilysozyme activity. Science 239, 1534-1536; and U.S. Pat. No. 4,816,567), by substituting human CDRs with corresponding rodent CDRs. Accordingly, humanized antibodies are chimeric antibodies, wherein substantially less than an intact human variable domain has been substituted by the corresponding sequence from a non-human species. In practice, humanized antibodies may be typically human antibodies in which some CDR residues and possibly some framework residues are substituted by residues from analogous sites in rodent antibodies.

Human antibodies can also be produced using various additional techniques known in the art, including phage-display libraries (Hoogenboom, H. R. and Winter, G. (1991). By-passing immunisation. Human antibodies from synthetic repertoires of germline VH gene segments rearranged in vitro. J Mol Biol 227, 381-388; Marks, J. D. et al. (1991). By-passing immunization. Human antibodies from V-gene libraries displayed on phage. J Mol Biol 222, 581-597; Cole et al. (1985), Monoclonal Antibodies and Cancer Therapy, Alan R. Liss, Inc., pp. 77-96; and Boerner, P. et al. (1991). Production of antigen-specific human monoclonal antibodies from in vitro-primed human splenocytes. J Immunol 147, 86-95). Humanized antibodies can also be created by introducing sequences encoding human immunoglobulin loci into transgenic animals, e.g., into mice in which the endogenous immunoglobulin genes have been partially or completely inactivated. Upon antigenic challenge, human antibody production is observed in such animals which closely resembles that seen in humans in all respects, including gene rearrangement, chain assembly, and antibody repertoire. Ample guidance for practicing such an approach is provided in the literature of the art (for example, refer to: U.S. Pat. Nos. 5,545,807; 5,545,806; 5,569,825; 5,625,126; 5,633,425; and 5,661,016; Marks, J. D. et al. (1992). By-passing immunization: building high affinity human antibodies by chain shuffling. Biotechnology (N.Y.) 10(7), 779-783; Lonberg et al., 1994. Nature 368:856-859; Morrison, S. L. (1994). News and View: Success in Specification. Nature 368, 812-813; Fishwild, D. M. et al. (1996). High-avidity human IgG kappa monoclonal antibodies from a novel strain of minilocus transgenic mice. Nat Biotechnol 14, 845-851; Neuberger, M. (1996). Generating high-avidity human Mabs in mice. Nat Biotechnol 14, 826; and Lonberg, N. and Huszar, D. (1995). Human antibodies from transgenic mice. Int Rev Immunol 13, 65-93).

After antibodies have been obtained, they may be tested for activity, for example via enzyme-linked immunosorbent assay (ELISA).

Non-Proteinaceous Agents

Numerous non-proteinaceous agents are known in the art as anti-amyloid agents. Typically, such compositions are of an aromatic nature, as explained hereinabove.

One example of a group of compounds which can be used in accordance with the present invention are phenol-containing compounds (see for example, PCT Appl. WO 2005/027901) such as having the general Formula I:

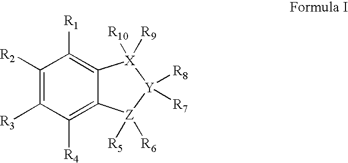

a pharmaceutically acceptable salt thereof or a prodrug thereof,

wherein:

X, Y and Z are each independently selected from the group consisting of carbon, oxygen, sulfur, CR₁₁R₁₂ or R₁₃R₁₄C—CR₁₅R₁₆, provided that at least one of X, Y and Z is oxygen or sulfur;

R₁-R₁₆ are each independently selected from the group consisting of hydrogen, lone pair electrons, hydroxy, alkyl, cycloalkyl, phenyl, alkoxyphenyl, thioalkoxyphenyl, aryloxyphenyl, thioaryloxyphenyl, carboxyphenyl, thiocarboxyphenyl, phenol, hydroxyphenol, dihydroxyphenol, aryl, alkenyl, alkynyl, heteroaryl, heteroalicyclic, halo, alkoxy, aryloxy, thiohydroxy, thioalkoxy, thioaryloxy, C-carboxy, O-carboxy, thiocarboxy, carbonyl, oxo, thiocarbonyl, sulfinyl, and sulfonyl, or absent, or, alternatively, at least two of R-R₄ and/or at least two of R₅-R₁₆ form at least one five- or six-membered aromatic, heteroaromatic, alicyclic or heteroalicyclic ring,

whereas:

at least one of R₁-R₄ is selected from the group consisting of hydroxy, alkoxy, aryloxy, thiohydroxy, thioalkoxy, thioaryloxy, O-carboxy and β-thiocarboxy; and/or

at least one of R₅-R₁₆ comprises phenol, alkoxyphenyl, thioalkoxyphenyl, aryloxyphenyl, thioaryloxyphenyl, carboxyphenyl, thiocarboxyphenyl hydroxyphenol, and dihydroxyphenol,

The compounds according to the present invention therefore include at least one phenol moiety (preferably at least two phenol moieties). As is further defined hereinbelow, each of the phenol moieties can be either unsubstituted or substituted, preferably by one or more hydroxy groups, thus being hydroxyphenol or dihydroxyphenol. Each of the phenol moieties can be present within the compounds of the present invention either per se, namely as a hydroxyphenyl moiety, or as an alkoxylated or carboxylated phenol moiety, namely, as an alkoxyphenyl or carboxyphenyl moiety, as is delineated hereinunder.

An “alkenyl” group refers to an alkyl group, as defined hereinabove, which consists of at least two carbon atoms and at least one carbon-carbon double bond.

An “alkynyl” group refers to an alkyl group, as defined hereinabove, which consists of at least two carbon atoms and at least one carbon-carbon triple bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, carbonyl, thiocarbonyl, ester, ether, carboxy, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, sulfonamido, trihalomethanesulfonamido, guanyl, guanidino, and amino, as these terms are defined herein.

A preferred example of a substituted aryl, according to the present invention is phenol.

As used herein, the term “phenol” refers to a phenyl substituted by an hydroxy group. The phenol group may be substituted or unsubstituted. When substituted, the substituent group can be, for example, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, carbonyl, thiocarbonyl, ester, ether, carboxy, thiocarboxy, thioether, thiocarbamate, urea, thiourea, O-carbamyl, N-carbamyl, β-thiocarbamyl, N-thiocarbamyl, C-amido, N-amido, C-carboxy, O-carboxy, sulfonamido, trihalomethanesulfonamido, guanyl, guanidino, and amino, as these terms are defined herein.

A preferred example of a substituted phenol, according to the present invention, is hydroxyphenol.

As used herein, the term “hydroxyphenol”, which also encompasses the term “dihydroxyphenol” refers to a phenol, as defined hereinabove, which is further substituted by one or more additional hydroxy groups. The additional hydroxy groups can be at the para, ortho and/or meta positions with respect to the hydroxy group of the phenol. The hydroxyphenol may be additionally substituted or unsubstituted. When substituted, the substituent group can be, for example, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, carbonyl, thiocarbonyl, ester, ether, carboxy, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, sulfonamido, trihalomethanesulfonamido, guanyl, guanidino, and amino, as these terms are defined herein.

Another preferred examples of a substituted aryl, according to the present invention, include alkoxyphenyl, thioalkoxyphenyl, aryloxyphenyl and thioaryloxyphenyl.

As used herein, the term “alkoxyphenyl” refers to a phenyl substituted by an alkoxy group, as defined herein. A representative example of an alkoxy group is methoxy.

The term “thioalkoxyphenyl” refers to a phenyl substituted by a thioalkoxy group, as defined herein.

The term “aryloxyphenyl” refers to a phenyl substituted by an aryloxy group, as defined herein.

The term “thioaryloxyphenyl” refers to a phenyl substituted by a thioaryloxy group, as defined herein.

Each of the alkoxyphenyl, thioalkoxyphenyl, aryloxyphenyl and thioaryloxyphenyl groups may be substituted or unsubstituted. When substituted, the substituent group can be, for example, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, carbonyl, thiocarbonyl, ester, ether, carboxy, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, sulfonamido, trihalomethanesulfonamido, guanyl, guanidino, and amino, as these terms are defined herein.

Preferred substituents of the alkoxyphenyl, thioalkoxyphenyl, aryloxyphenyl and thioaryloxyphenyl groups include alkoxy, thioalkoxy, aryloxy and/or thioaryloxy groups, such that examples of preferred substituted alkoxyphenyl, thioalkoxyphenyl, aryloxyphenyl and thioaryloxyphenyl include dialkoxyphenyl, dithioalkoxyphenyl, diaryloxyphenyl and dithioaryloxyphenyl, and any other combination.

As used herein, the term “dialkoxyphenyl”, refers to an alkoxyphenyl, as defined hereinabove, which is further substituted by one or more additional alkoxy groups. The additional alkoxy groups can be at the para, ortho and/or meta positions with respect to the alkoxy group of the alkoxyphenyl.

The term “dithioalkoxyphenyl”, refers to a thioalkoxyphenyl, as defined hereinabove, which is further substituted by one or more additional thioalkoxy groups. The additional thioalkoxy groups can be at the para, ortho and/or meta positions with respect to the thioalkoxy group of the thioalkoxyphenyl.

The term “diaryloxyphenyl”, refers to an aryloxyphenyl, as defined hereinabove, which is further substituted by one or more additional aryloxy groups. The additional aryloxy groups can be at the para, ortho and/or meta positions with respect to the aryloxy group of the aryloxyphenyl.

The term “dithioaryloxyphenyl”, refers to a thioaryloxyphenyl, as defined hereinabove, which is further substituted by one or more additional thioaryloxy groups. The additional thioaryloxy groups can be at the para, ortho and/or meta positions with respect to the thioaryloxy group of the thioaryloxyphenyl.

Each of the dialkoxyphenyl, dithioalkoxyphenyl, diaryloxyphenyl and dithioaryloxyphenyl may be additionally substituted or unsubstituted. When substituted, the substituent group can be, for example, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, carbonyl, thiocarbonyl, ester, ether, carboxy, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, sulfonamido, trihalomethanesulfonamido, guanyl, guanidino, and amino, as these terms are defined herein.

Another preferred examples of a substituted aryl, according to the present invention, include carboxyphenyl and thiocarboxyphenyl.

As used herein, the term “carboxyphenyl” refers to a phenyl substituted by an O-carboxy group, as defined herein. A representative example of an O-carboxy group is O-acetoxy.

The term “thiocarboxyphenyl” refers to a phenyl substituted by a thiocarboxy group, as defined herein.

The carboxyphenyl and the thiocarboxyphenyl may be substituted or unsubstituted. When substituted, the substituent group can be, for example, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, carbonyl, thiocarbonyl, ester, ether, carboxy, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, sulfonamido, trihalomethanesulfonamido, guanyl, guanidino, and amino, as these terms are defined herein.

Preferred substituents include additional O-carboxy or thiocarboxy groups, such that examples of preferred substituted carboxyphenyl and thiocarboxyphenyl include dicarboxyphenyl and dithiocarboxyphenyl.

As used herein, the term “dicarboxyphenyl”, refers to a carboxyphenyl, e.g., acetoxyphenyl, as defined hereinabove, which is further substituted by one or more additional carboxy groups. The additional carboxy groups can be at the para, ortho and/or meta positions with respect to the carboxy group of the carboxyphenyl.

The term “dithiocarboxyphenyl”, refers to a thiocarboxyphenyl, as defined hereinabove, which is further substituted by one or more additional thiocarboxy groups. The additional thiocarboxy groups can be at the para, ortho and/or meta positions with respect to the thiocarboxy group of the thiocarboxyphenyl.

Each of the dicarboxyphenyl and dithiocarboxyphenyl may be additionally substituted or unsubstituted. When substituted, the substituent group can be, for example, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, carbonyl, thiocarbonyl, ester, ether, carboxy, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, sulfonamido, trihalomethanesulfonamido, guanyl, guanidino, and amino, as these terms are defined herein.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, carbonyl, thiocarbonyl, ester, ether, carboxy, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, sulfonamido, trihalomethanesulfonamido, guanyl, guanidino, and amino, as these terms are defined herein.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, lone pair electrons, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, carbonyl, thiocarbonyl, ester, ether, carboxy, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, sulfonamido, trihalomethanesulfonamido, guanyl, guanidino, and amino, as these terms are defined herein. Representative examples are piperidine, piperazine, tetrahydrofurane, tetrahydropyrane, morpholino and the like.

An “alkoxy” group refers to both an —O-alkyl and an —O-cycloalkyl group, as defined herein.

An “aryloxy” group refers to both an —O-aryl and an —O-heteroaryl group, as defined herein.

An “oxo” group refers to an ═O group.

A “carbonyl” group refers to a —C(═O)—R′ group, where R′ is hydrogen, alkyl, alkenyl, cycloalkyl, aryl, heteroaryl (bonded through a ring carbon) or heteroalicyclic (bonded through a ring carbon) as defined herein.

A “thiocarbonyl” group refers to a —C(═S)—R′ group, where R′ is as defined herein for R′.

An “O-carboxy” group refers to a R″C(═O)—O— group, where R″ is as defined herein.

A “sulfinyl” group refers to an —S(═O)—R″ group, where R″ is as defined herein.

A “sulfonyl” group refers to an —S(═O)₂—R″ group, where R″ is as defined herein.

A “trihalomethyl” group refers to a —CX group wherein X is a halo group as defined herein.

A “trihalomethanesulfonyl” group refers to a X₃CS(═O)₂— group wherein X is a halo group as defined herein.

A “S-sulfonamido” group refers to a —S(═O)₂—NR′R″ group, with R′ and R″ as defined herein.

A “N-sulfonamido” group refers to n R′S(═O)₂—NR″ group, where R′ and R″ are as defined herein.

A “trihalomethanesulfonamido” group refers to an X₃CS(═O)₂NR′— group, where R′ and X are as defined herein.

An “O-carbamyl” group refers to an —OC(═O)—NR′R″ group, where R′ and

R″ are as defined herein.

An “N-carbamyl” group refers to an R″OC(═O)—NR′— group, where R′ and

R″ are as defined herein.

An “β-thiocarbamyl” group refers to an —OC(═S)—NR′R″ group, where R′ and

R″ are as defined herein.

An “N-thiocarbamyl” group refers to an R″OC(═S)NR′— group, where R′ and

R″ are as defined herein.

An “amino” group refers to an —NR′R″ group where R′ and R″ are as defined herein.

A “C-amido” group refers to a —C(═O)—NR′R″ group, where R′ and R″ are as defined herein.

An “N-amido” group refers to an R′C(═O)—NR″ group, where R′ and R″ are as defined herein.

An “urea” group refers to an —NR′C(═O)—NR″R′″ group, where R′ and R″ are as defined herein and R′″ is defined as either R′ or R″.

A “guanidino” group refers to an —R′NC(═N)—NR″R′″ group, where R′, R″ and R′″ are as defined herein.

A “guanyl” group refers to an R′R″ NC(═N)— group, where R′ and R″ are as defined herein.

An “azo” group refers to a —N═N group.

The term “phosphonyl” describes a —O—P(═O)(OR′)(OR″) group, with R′ and R″ as defined hereinabove.

The term “phosphinyl” describes a —PR′R″ group, with R′ and R″ as defined hereinabove.

Preferred phenol-containing compounds according to the present invention therefore include, for example, phenol red and analogs thereof, such that in the Formula above X is carbon; Y is oxygen; Z is carbon or sulfur; and at least one of R₅ and R₆ is oxo, as this term is defined hereinabove. Such compounds include a heteroalicyclic ring, fused with phenyl, and further substituted by one or more phenol or phenyl groups, such that at least one of R₅-R₁₀ is phenol or hydroxyphenol, as defined hereinabove. Such compounds in which at least one, and preferably two, of R₅-R₁₀ are hydroxyphenol include, for example, pyrocatechol violet and analogs thereof.

Compounds in this category, in which Z is sulfur, are typically phenol red analogs, whereas compounds in which Z is carbon are typically phenolphthaleine analogs.

Even more preferred compounds according to the present invention, include compounds having the Formula above, in which X is carbon; Y is R₁₃R₁₄C—CR₁₅R₁₆; and Z is oxygen. Such compounds therefore include a tetrahydropyrane ring fused to phenyl.

Preferred examples of compounds in this category include analogs and derivatives of catechins such as, for example, analogs and derivatives of epicatechin, epigallocatechin, epigallocatechin gallate and the like, all include two hydroxy group at the R₁ and R₃ positions and a hydroxyphenol or dihydroxyphenol group, directly or indirectly attached to the tetrahydropyrane ring, at one or more of the R₁₃-R₁₆ positions in the Formula above.

Additional preferred examples of these compounds include an oxidized tetrahydropyrane ring fused to a phenyl, such that R₉ is oxo; and R₁₀ is absent.

Further additional preferred compounds in this category include tocopherol and analogs thereof, which include one or more alkyl groups at the R₁₃-R₁₆ positions, whereby the alkyl groups can include lower alkyls (e.g., methyl) and/or alkyls having more than 8 carbon atoms.

Further according to the present invention, each of the compounds described above can further be in a dimeric form. Such a dimeric form includes two moieties having the Formula above, attached therebetween via R₁-R₁₆, directly or indirectly.

Examples of phenol-containing compounds which can be used in accordance with the present invention therefore include, but are not limited to, phenol red, pyrocatechol violet, phenolphthaleine, catechin, epigallocatechin gallate, epicatechin gallate, epicatechin, epigallocatechin, eriodictyol, quercetin, procyanidin, hydroxyphenyl, tocopherol, bromophenol red, analogs thereof, derivatives thereof and any combination thereof.

The presently most preferred phenol-containing compounds according to the present invention are phenol red, pyrocatechol violet and compounds of the catechin gallate family (for further details see the Examples section which follows).

However, additional preferred compounds which can be used in accordance with the present invention include the mono-, di-, tri- and tetra-alkoxy (e.g., methoxy) or carboxy (e.g., acetoxy) derivatives of the compounds listed above. Such derivatives are meant to include compounds in which one or more of the hydroxy groups in the phenol or hydroxyphenol moieties are derivatized by, e.g., an alkyl or acyl group, is resulting in an alkoxyphenyl moiety, a dialkoxyphenyl moiety, a carboxyphenyl moiety or a di-carboxyphenyl moiety.

Such a derivatization of the hydroxy groups, which results in the replacement of one or more of the phenol moieties by an alkoxyphenyl moiety, a dialkoxyphenyl moiety, a carboxyphenyl moiety or a di-carboxyphenyl moiety, as well as analogs thereof (e.g., aryloxyphenyl, thioalkoxyphenyl, and the like, as is detailed hereinabove) is highly advantageous since it reduces the hydrophilic nature of the compounds and thus enhances their absorption in the intestines.

As is well known in the art, hydrophilic compounds are typically characterized by relatively low absorption due to poor permeability across human intestinal epithelial. Due to these low absorption parameters, treatment with hydrophilic compounds requires the administration of high doses, when administered orally. Hence, reducing the hydrophilic nature of the compounds described above provides for enhanced absorption thereof, particularly in the intestines, and enables an effective oral administration thereof. The effect of reducing the hydrophilic nature of compounds on their absorption was clearly shown in several models, including the Caco-2 cells and parallel artificial membrane permeation assay (PAMPA). These studies demonstrated that increased hydrophobicity significantly improves the permeability of small organic compounds [Ano (2004) Bioorg Med. Chem. 12:257-264; Ano (2004) 12: 249-255].

Representative examples of such derivatives include, but are not limited to, methoxy phenol red and acetoxy phenol red, in which one phenol moiety in phenol red is replaced by a methoxyphenyl or an acetoxyphenyl moiety, respectively, and dimethoxy phenol red and diacetoxy phenol red, in which the two phenol moieties in phenol red are replaced by two methoxyphenyl or acetoxyphenyl moieties, respectively.

Of a particular importance are the mono derivatives of phenol red, namely, methoxy phenol red and acetoxy phenol red and analogs thereof. These mono derivatives simultaneously provide for (i) enhanced inhibition activity due to the presence of hydroxy groups; (ii) enhanced oral bioavailability due a partial hydrophilic nature thereof; and (iii) enhanced absorption due to a partial hydrophobic nature thereof, as is detailed hereinabove.

Hence, the phenol red mono derivatives of the present invention, by combining enhanced inhibition activity, enhanced oral bioavailability and enhanced absorption, are highly advantageous.
Another group of compounds which can be used in accordance with the present invention are indole-derivatives (see for example, U.S. Pat. Appl. No. 60/649,574), such as having the general formula:

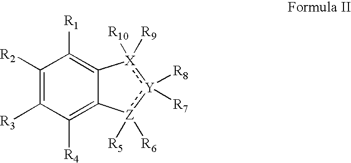

a pharmaceutically acceptable salt thereof, or a prodrug thereof,
wherein:

the dashed line denotes a double bond either between X and Y, or, between Y and Z;

X, Y and Z are each independently selected from the group consisting of carbon and nitrogen, whereas at least one of X, Y, and Z is nitrogen; and

R₁-R₁₀ are each independently selected from the group consisting of hydrogen, lone pair electrons, hydroxy, alkyl, cycloalkyl, phenyl, phenol, hydroxyphenol, dihydroxyphenol, aryl, alkenyl, alkynyl, heteroaryl, heteroalicyclic, halo, alkoxy, aryloxy, thiohydroxy, thioalkoxy, thioaryloxy, C-carboxy, O-carboxy, thiocarboxy, carbonyl, oxo, thiocarbonyl, sulfinyl, and sulfonyl, or absent, or, alternatively, at least two of R₁-R₁₀ form at least one five- or six-membered aromatic, heteroaromatic, alicyclic or heteroalicyclic ring.

Thus, preferred indole-derived compounds which conform to the above illustratively described general formula, and which can be used for use in accordance with the present invention, are therefore indole derivatives, being compounds having an aromatic ring fused to a heterocyclic ring having at least one nitrogen atom. The parent compound, indole, is a heteroaromatic compound having a phenyl ring fused to a pyrrole ring and thus comprises a completely conjugated pi-electron system.

However, an indole derivative, according to the present invention, encompasses any aromatic moiety that is fused to a heterocyclic ring containing one or more nitrogen atoms (for example, one, two or three nitrogen atoms). Depending of the location of the pi-electrons of the double bond (between X and Y or Y and Z, see, the formula above) and the nature of the ring atoms (carbon and/or nitrogen), the electronic structure of an indole derivative according to the present invention can include either a partially or completely conjugated pi-electron system.

Thus, an indole derivative, according to the present invention, encompasses, for example, substituted or unsubstituted indoles, substituted or unsubstituted purines, substituted or unsubstituted carbazoles and substituted or unsubstituted phenyl ring fused to a substituted or unsubstituted imidazole, pyrazole, thiazine, and the like, with substituted or unsubstituted indoles being the presently preferred indole derivatives.

Thus, preferred compounds which can be used for use in accordance with the present invention, are compounds which have the above illustratively described general formula, wherein each of X and Y is carbon, and Z is nitrogen, whereby the double bond (dashed line) is preferably between X and Y.

Further preferred compounds for use in accordance with the present invention, are compounds which have the above illustratively described general formula, wherein one or more of R₁-R₁₀ comprises a hydroxy group. In such compounds, the one or more hydroxy groups are directly or indirectly attached to the indole derivative skeleton, such that at least one of R₁-R₁₀ is either hydroxy or, for example, a hydroxyalkyl, as defined hereinabove.

Particularly preferred compounds which can be used for use in accordance with the present invention, are indoles substituted by a hydroxy group and are therefore compounds which have the above illustratively described general formula, wherein

each of X and Y is carbon, and Z is nitrogen, the double bond (dashed line) is between X and Y, and at least one of R₁-R₁₀ is a hydroxy group. Preferably, in such hydroxy group containing compounds, at least one of R₁, R₃, R₄, and R₉ is a hydroxy group, and more preferably, R₁ or R₉ is a hydroxy group. More preferably, in such hydroxy group containing compounds, each of R₂-R₅ and R₇ is hydrogen and R₆, R₈ and R₁₀ are absent.

A representative example of such a hydroxy containing compound is 3-hydroxyindole, such that in the general formula, R₁ is hydrogen and R₉ is the hydroxy group. Another representative example of such a hydroxy containing compound is 4-hydroxyindole, such that in the general formula, R₁ is the hydroxy group and R₉ is hydrogen.

Additional particularly preferred compounds which can be used for use in accordance with the present invention, are indoles substituted by one or more hydroxyalkyl groups and are therefore compounds which have the above illustratively described general formula, wherein each of X and Y is carbon, and Z is nitrogen, the double bond (dashed line) is between X and Y, and at least one of R₁-R₁₀ is a hydroxyalkyl. Preferably, in such hydroxyalkyl containing compounds, at least one of R₇ and R₉ is a hydroxyalkyl. More preferably, in such hydroxyalkyl containing compounds, each of R₁-R₅ is hydrogen, and R₆, R₈ and R₁₀ are absent. More preferably, in such hydroxyalkyl containing compounds, at least one of R₇ and R₉ is a hydroxymethyl type of hydroxyalkyl.

A representative example of such a hydroxyalkyl containing compound is indole-3-carbinol (3-hydroxymethyl indole), such that in the general formula, R₇ is hydrogen and R₉ is a hydroxymethyl.

Examples of other non-protein anti-amyloid agents which can be used in accordance with the present invention include, but are not limited to, nicotine [Salomon (1996) Biochemistry 35:13568-13578], acridine and acridine orange, Congo red, methylene blue, tetracycline and Thioflavin-T [each of which described by Aitken (2003) Biochem. J. 374:779-784] and non-steroidal anti-inflammatory drugs as listed in Table 3 below.

TABLE 3
NSAIDs- nonsteroidal anti-inflammatory drugs

| | |
|---|---|
| DRUG | BRAND NAME(S) |

Traditional NSAIDs

| | |
|---|---|
| Diclofenac potassium | Cataflam |
| Diclofenac sodium | Voltaren, Voltaren XR |
| Diclofenac sodium | Arthrotec |
| with misoprostol | |
| Diflunisal | Dolobid |
| Etodolac | Lodine, Lodine XL |
| Fenoprofen calcium | Nalfon |
| Flurbiprofen | Ansaid |
| Ibuprofen | Motrin, Advil, Motrin IB, Nuprin |
| Indomethacin | Indocin Indocin SR |
| Ketoprofen | Orudis Oruvail Actron, Orudis, KT |
| Meclofenamate sodium | Meclomen |
| Mefenamic acid | Ponstel |
| Meloxicam | Mobic |
| Nabumetone | Relafen |
| Naproxen | Naprosyn, Naprelan |
| Naproxen sodium | Anaprox, Aleve |
| Oxaprozin | Daypro |
| Piroxicam | Feldene |
| Sulindac | Clinoril |
| Tolmetin sodium | Tolectin |

COX-2 Inhibitors

| | |
|---|---|
| Celecoxib | Celebrex |
| Rofecoxib | Vioxx |
| Valdecoxib | Bextra |

Salicylates
Acetylated Salicylates

| | |
|---|---|
| Aspirin | Anacin, Ascriptin, Bayer, Bufferin, |
| | Ecotrin, Excedrin tablets |

Nonacetylated Salicylates

| | |
|---|---|
| Choline and magnesium | CMT, Tricosal, Trilisate |
| salicylates | |
| Choline salicylate | Arthropan |
| (liquid only) | |
| Magnesium salicylate | Magan, Mobidin, Mobogesic, |
| | Arthritab, Bayer Select, |
| | Doan's Pill |
| Salsalate | Amigesic, Anaflex 750, Disalcid, |
| | Marthritic, Mono-Gesic, Salflex, |
| | Salsitab |
| Sodium salicylate | |
| (Available as generic only) | |

Accordingly, the anti-amyloid agents of the present invention (also referred to as compounds of the present invention, described hereinabove) can be provided to the subject per se, or as part of a pharmaceutical composition where it is mixed with a pharmaceutically acceptable carrier.

As used herein a ‘pharmaceutical composition’ refers to a preparation of one or more of the active ingredients described herein with other chemical components such as physiologically suitable carriers and excipients. The purpose of a pharmaceutical composition is to facilitate administration of a compound to the subject treated.

Herein the term ‘active ingredient’ refers to the compound, which is accountable for the biological effect.

Hereinafter, the phrases ‘physiologically acceptable carrier’ and ‘pharmaceutically acceptable carrier’ which may be interchangeably used refer to a carrier or a diluent that does not cause significant irritation to the subject and does not abrogate the biological activity and properties of the administered compound. Preferred carriers of the pharmaceutical composition of the present invention include, but are not limited to, polyethylene glycol (PEG), a biocompatible polymer with a wide range of solubility in both organic and aqueous media (Mutter et al. (1979).

Herein the term ‘excipient’ refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in ‘Remington's Pharmaceutical Sciences,’ Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

According to another aspect of the present invention, there is provided an article-of-manufacture including a packaging material and a pharmaceutical composition identified for treating amyloid associated diseases being contained within the packaging material, the pharmaceutical composition including, as an active ingredient, the compound described hereinabove, and a pharmaceutically acceptable carrier.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

It will be appreciated that the use of other antibiotic agents can be used in combination with the agents of the present invention to increase therapeutic efficacy thereof.

It will be appreciated that medical devices are commonly infected with opportunistic bacteria and other infectious micro-organisms (e.g., fungi), in some cases necessitating the removal of implantable devices. Such infections can also result in illness, long hospital stays, or even death. The prevention of biofilm formation and infection medical devices is therefore highly desirous.

Thus, the present invention also contemplates medical devices in which the above-described anti-amyloid agent is attached thereto.

Examples of medical devices which can be used in accordance with the present invention include, but are not limited to, clamps, valves, intracorporeal or extracorporeal devices (e.g., catheters), temporary or permanent implants, stents, vascular grafts, anastomotic devices, aneurysm repair devices, embolic devices, and implantable devices (e.g., orthopedic implants) and the like. Other devices which can be used in accordance with the present invention are described in U.S. Pat. Appl. No. 20050038498.

The availability of new antibiotic agents, which are capable of characterizing a pathogen based on structural properties (i.e., amyloid formation) allows use thereof in laboratory procedures.

Thus, according to another aspect of the present invention there is provided a method of typing a pathogen.

The method is effected by monitoring an alteration (e.g., decreased) in growth and/or infectivity of the pathogen in the presence of the anti-amyloid agent, thereby typing the pathogen.

Preferably monitored are pathogens present in a biological sample.

As used herein the phrase “biological sample” refers to a biological fluid such as blood, serum, plasma, lymph, bile fluid, urine, saliva, sputum, synovial fluid, semen, tears, cerebrospinal fluid, bronchioalveolar large fluid, ascites fluid, pus, tissue sections, cell cultures and conditioned medium and the like in which the pathogen may be present.

Methods of determining growth and infectivity of microorganisms are well known in the art and may be chosen according to the nature of the examined pathogen (i.e., bacteria or fungi).

It should be noted that final identification of the pathogen may necessitate use of other antibiotic agents which are well known in the art, as well as morphological analyses.

The formation of typical amyloid by bacteria may also facilitate the search for therapeutic agents to treat amyloid disease. As amyloid fibril formation by non-homologous protein appears to share common characteristic features, and small molecule amyloid inhibitors (such as Congo Red or polyphenol catechins) show cross-reactivity, curli-related amyloid formation may serve as a model system to study potential amyloid inhibitors. The low-cost and reproducibility of the bacterial amyloid formation may overcome limitation set by the high cost and seeding variability of synthetic amyloidogenic polypeptide and allow high throughput screen of candidate inhibitors.

Thus, the present invention also envisages a method of identifying an anti-amyloid agent, the method comprising: (a) contacting molecules with an amyloid forming pathogen: and (b) identifying at least one molecule of said molecules capable of altering amyloid formation of the amyloid forming pathogen, thereby identifying the anti-amyloid agent.

Identification of an alteration in amyloid formation by the amyloid forming pathogen can be effected by any method known in the art for detecting amyloid aggregates [e.g., congo red binding, Thioflavin binding, circular dichroism (CD), TEM analysis described in length in the Examples section which follows] as well as by identifying the effect thereof on biofilm formation (e.g., fibronectin binding, internalization assay).

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Procedures

Peptide synthesis—Peptides listed in Table 4 below were synthesized using solid-phase methods was performed by Peptron Inc. (Taejeon, Korea). Identity of the peptides was confirmed by ion spray mass-spectrometry and the purity of the peptides was confirmed by reverse phase high-pressure liquid chromatography.

TABLE 4

| | |
|---|---|
| Peptide | SEQ ID NO. |

| | |
|---|---|
| PQGGYQQYN | 1 |
| PHGGGWGQ | 2 |
| QFGGGN | 3 |
| QAGGGN | 4 |
| QHGGGN | 5 |
| NHQGGG | 6 |
| HGQGNG | 7 |
| GQNHGG | 8 |
| PQFGGGNP | 9 |
| PPQFGGGNPP | 10 |
| -QFGGGNPP | 11 |

Congo red binding—Bacterial inoculums were grown in 6 ml LB medium for 5-7 hours at 37° C. in the presence or absence of 0.3 mM peptide inhibitor, until the cultures reached A₆₀₀ of approximately 1.2. Each culture was then pelleted (1 min/13200×g) and resuspended in 6 ml saline (0.8% NaCl). Three double dilutions were performed for each culture and bacterial concentrations were determined at A₆₀₀. Each dilution was divided into 3 microtubes (1 ml for each) and pelleted by centrifugation for 2 min/13200×g. A 0.004% solution of CR (in saline) was prepared and optical density at 487 nm was measured against a saline background. One thousand, 500 and 200 μl of CR solution were then added to each dilution triplets, the bacteria were well resuspended in the dye and left for 10 min incubation at RT. Next, a second centrifugation (2 min/13200×g) was performed and the supernatants containing the remaining dye were recovered. The optical density at 487 nm of each supernatant was determined and compared to the initial absorbance. Binding units were calculated as follows:

$$unit = \frac{Δ {OD}_{487}}{{A}_{600} ( bacteria )} \times 1000$$

TEM analysis—In all examined cases, peptides were dissolved in water to a concentration of 10 to 50 mg/ml and incubated at room-temperature (RT). Fresh stocks were prepared for each and every experiment. Fibril formation was assessed after at least 3 days using 10 μl of sample aged at RT and placed on 400-mesh copper grid. Following 1 minute, excess fluid was removed, and the grid was then negatively stained with 10 μl of 2% uranyl-acetate in water. Following 2 minutes, excess fluid was removed from the grid. Samples were viewed in JEOL 1200 EX transmission electron microscope. In the experiments shown, 50 mg/ml peptides were used and micrographs were taken following 1 week. Bar represents 100 nm.

Circular dichroism (CD) spectra and Copper binding—Circular Dichroism (CD) spectra, 250-800 nm, of the curli QHGGGN hexapeptide repeat in the absence and presence were preformed. Changes in CD signals with increasing amounts of Cu²⁺ were measured at 590 nm, indicating equimolar binding stoichiometry. The titration of metal ions to the oligopeptide was performed using small aliquots from stock aqueous solutions of 25 mM CuCl₂-2H₂O. CD spectrum (250-800 nm) of QHGGGN (0.5 mM, pH 7.5) was measured with the addition of Cu²⁺ in increments of 0.0125 mM (as a minimum) of CuCl₂ (0.5 μl) from 0 up to 0.65 mM. 50 mM HEPES pH 7.5 buffer was used for CD studies.

Fibronectin binding—Binding was quantified using the protocol described by Gophna et al (32). Briefly, wells of microtiter plates were coated with 100 μl of 5 μg/ml solution of bovine fibronectin (Sigma). The two strains of bacteria were grown to mid-exponential phase (A₆₀₀, 0.5-0.6) with the various peptides in LB, harvested and suspended in saline. Wells were blocked with 200 μl milk (3%) for 2 hours at RT. Two hundreds micro-liter saline containing 5×10⁸ cells/ml bacteria (A₆₀₀, 0.9) were added to the wells with 0.3 mM of the suitable peptide and were incubated at RT for 3 h. Wells were washed twice with 200 μl PBS-T (0.05% Tween) and once with 200 μl PBS. The optical density of adherent bacteria was determined by the use of a microplate reader (ELISA) (A₄₀₅), subtracting the optical density of an empty coated well.

Internalization assay—Epithelial cells were grown overnight in 24-well plates to 80% confluence (˜10⁵ cells per well). Bacteria were inoculated in LB broth and grown at 37° C. for 16 h, diluted 100-fold, and grown to mid-exponential phase (A₆₀₀, 0.4) in the presence or absence of 0.4 mM peptide inhibitor. Bacteria were then diluted to A₆₀₀, 0.1 in LB and 20 μl were added to each well with epithelial monolayer for 3 h incubation at 37° C. Presence of 0.4 mM inhibitor peptide was kept in the appropriate wells. Cells were washed once with PBS, pH 7.3, and PBS containing 100 μg/ml gentamicin was added. After 1.5 h incubation, the cells were washed twice in PBS, and lysed by 5 min incubation with ice-cold 0.5% triton X-100. Appropriate bacterial dilutions were plated to determine the number of viable internalized bacteria. E. coli XL1-Blue was maintained on Luria-Bertani (LB) agar and E. coli XL1-Blue(pMRInv) was maintained on LB agar containing 30 μg/ml kanamycin. HEK293 cell line (purchased from the ATCC) was cultivated in Dulbecco's Modified Eagle Medium (DMEM) supplemented with 2 mM L-glutamine, 10% heat-inactivated fetal calf serum (FCS) and antibiotics (100 U/ml Penicillin, 100 μg/ml Streptomycin). Prior to bacterial infection, cells were added to 96-well tissue culture plates and incubated for 24 hours at 37° C. in a 5% CO₂ incubator.

### Example 1

### Identification of Prion-Like Hexarepeats within Curlin Major Subunit

The sequence of the CsgA protein consists of internal homologies (17). While analyzing the sequence of the amyloid forming CsgA protein, short oligopeptide repeats were identified which share a general chemical composition similarity with yeast and animal prion protein repeats (FIG. 1a). All three groups of short (6-9 residues) representative consensus repeats, PQGGYQQYN (SEQ ID NO. 1), PHGGGWGQ (SEQ ID NO. 2) and QFGGGN (SEQ ID NO. 3), in the case of yeast prion, human prion and curlin repeats respectively, are characterized by aromatic residues in conjugation with glycines (typically multiple) and glutamine/asparagine residues. This is also the case for the immediate homolog of CsgA, the AgfA fimbrin subunit of Salmonella (NCBI accession no. AAC43599, 17). Thus, it is postulated that hydrogen bonds interaction between the amides chains (9, 19) in concert with aromatic interactions (19-24), along with the structural flexibility provided by the glycine residues, facilitate the process of oligomeric molecular recognition and specific self-assembly that eventually lead to the formation of amyloid fibrils.

### Example 2

### Self-Assembly Potential of Hexapeptide Repeats

To gain insight into the potential ability of the CsgA repeat to mediate the process of homo-recognition that leads to the formation of multi-protein aggregates, the ability of short peptides, corresponding to the repeats, to self-assemble into fibrillar structures was studied.

Transmission Electron microscopy (TEM) analysis had demonstrated the ability of NH₂-QFGGGN-COOH and NH₂-QHGGGN-COOH fragments to associate into well-ordered fibrillar structures ranging from 10 to 100 nm in diameter (FIG. 1b) in 3-6 days. The importance of the phenylalanine and histidine residues in the formation of fibrils was further substantiated by electron microscopy analysis of similar oligopeptides lacking the aromatic residue. To this end, the fibrillogenesis potential of the QAGGGN (SEQ ID NO. 4) fragment under the same conditions was studied (FIG. 1b). As is shown, no ordered structures could be detected under these experimental conditions. Indeed, only minor amounts of amorphous aggregates were observed even following 30 days of incubation.

In order to determine whether QAGGGN (SEQ ID NO. 4) interferes with the self-assembly of the repeats, a mixture of QFGGGN (SEQ ID NO. 3) and QAGGGN (SEQ ID NO. 4) peptides were analyzed for fibril formation.

Following a similar incubation period (i.e., 3-6 days), typical QFGGGN (SEQ to ID NO. 3) fibrils were observed, indicating QFGGGN recognition and assembly was not inhibited.

To study sequence-dependency of the aggregation potential of the peptides, the self-assembly ability of peptides with scrambled QHGGGN sequences was examined. All peptides exhibited aggregation potential (FIG. 1c). The NHQGGG (SEQ ID NO. 6) peptide formed small amount but highly ordered assemblies, similar to QHGGGN (SEQ ID NO. 5) and QFGGGN (SEQ ID NO. 3) peptides. In contrast, the HGQGNG (SEQ ID NO. 7) and GQNHGG (SEQ ID NO. 8) peptides were highly aggregative and much more abundant. However, while GQNHGG (SEQ ID NO. 8) peptide assembled into fibril-like structured, HGQGNG (SEQ ID NO. 7) peptide formed amorphous aggregates.

These results clearly indicate that the mode of aggregation is dependent on the arrangement of the sequence within the repeat. It is suggested that while the set of residues comprising the peptide repeats prompt aggregation, their intra-order within the repeats in concert with the order of the repeat within CsgA direct the formation of the highly aggregative and ordered curli amyloid fibers.

### Example 3

### Copper Binding Potential by Hexapeptide Repeat

The animal prion protein (PrP^{C}) was suggested to act as a copper-binding glycoprotein, where the Cu²⁺ binding site was identified within peptide repeat domain (25). Specific and equimolar binding of copper by a single peptide repeat was clearly demonstrated. This ability is assumed to be mediated by coordination of the imidazole moieties of the histidine residues (25).

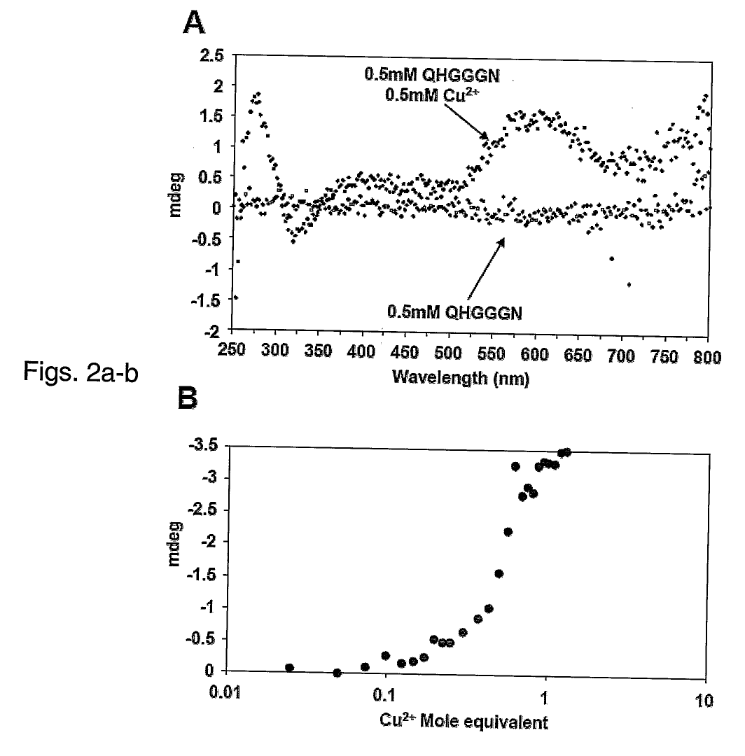

Circular dichroism (CD) spectroscopy was used to analysis the QHGGGN interaction with copper. As shown in FIG. 2a, increasing amounts of CuCl₂ altered both visible and UV regions of the CD spectra, consistent with both structural induction within the peptide motif and orientation of the copper ions within a rigid framework. Moreover, stoichiometric analysis of the CD signal of the copper is consistent with a single Cu²⁺ ion binding for each peptide molecule (FIG. 2b). The same equimolar binding stoichiometry was observed for the PrP octarepeat (26), suggesting a specific and well-coordinated binding which offers another structural link between the curlin and prion repeats.

The binding curve exhibits a typical sigmoid behavior at concentrations ranging in the hundreds of μM range. Thus, although the binding is clearly stoichiometric the affinity for a single repeat is not high. Yet, the binding to the full-length protein may show cooperative behavior. In spite of the clear binding, the presence of copper did not induce fibrillization of the QHGGGN peptide. This was deduced by inspecting QHGGGN peptides using electron microscopy, following 48 hours incubation with equimolar ratio of CuCl₂ in 50 mM HEPES, pH 7.5 at room-temperature. Nevertheless, when curli-expressing bacteria (E. coli XL1-Blue(pMRInv)) were grown for 12 hours at 37° C. in the presence of increasing amounts of copper (0, 10, 50 and 100 μM), the density of the bacterial biofilm, as observed by TEM, was higher in correlative manner (data not shown). However, this may be also due to the stress conditions imposed by the copper.

### Example 4

### The Design of Peptide Inhibitor of Curli Formation

To further study the potential role of the repeats in the process of self-assembly, the β-breaker methodology was used. This methodology is currently being used for the development of candidate drugs designated to inhibit the process of amyloid formation. This methodology, developed by Soto and co-workers (27-29), was achieved by the incorporation of a proline residue into a peptide fragment which included the recognition motif that mediates the process of self-assembly. In a similar a β-sheet breaker element was introduced into the identified oligopeptide repeat unit. It is suggested that the ability of such a hybrid peptide to interfere with the fibrillization process would further support the suggested role of this structural element.

To that purpose, several oligopeptides containing the QFGGGN motif conjugated to proline residues were initially screened. Proline residues were placed at one or either ends of the motif and inhibition of curli formation was evaluated using simple CR bonding assay (see below). Among NH₂-PQFGGGNP-COOH (SEQ ID NO: 9), NH₂-PPQFGGGNPP-COOH (SEQ ID NO: 10) and NH₂-QFGGGNPP-COOH (SEQ ID NO: 11), the presence of the latter peptide demonstrated lesser CR binding of curli expressing bacteria.

Thus, the oligopeptide QFGGGNPP was selected as a potential peptide inhibitor and examined its effect on the assembly of curli.

### Example 5

### Inhibition of Curli Formation

The curli proteins are secreted to the extra-cellular milieu and the whole polymerization process occurs outside the bacterium (30). This allows to assess the effect of the peptide directly in bacterial culture. The E. coli K-12 XL1-Blue strain harboring the cosmid (pMRInv) was used. Presence of several copies of this cosmid, which carries the entire csg gene cluster of E. coli strain 078, as well as a mutation in one of the regulatory genes of the system, causes its host to constitutively expresses curli at high levels. A wild-type E. coli K-12 XL1-Blue was used as a control. E. coli K-12 XL1-Blue was maintained on Luria-Bertani (LB) agar and E. coli K-12 XL1-Blue(pMRInv) was maintained on LB agar containing 30 μg/ml kanamycin. Curli expressing bacteria have several detectable properties by which curli formation level can be determined. First, curli is well characterized by its ability to bind the congo red (CR) dye (31). Second, curli expressing bacteria confer an exceptional ability to bind with high affinity several host molecules including fibronectin (32). And third, curli were suggested to have a role in bacteria pathogenesis, as curli fibers of E. coli were demonstrated to mediate internalization of bacteria by eukaryotic cells (33).

Congo Red Binding

In order to assess the effect of the putative recognition motif based inhibitor on curli formation (see Example 4, above), the E. coli K-12 strain XL1-Blue bacteria harboring the pMRInv cosmid was used (33-34). Inhibition was examined by directly applying the putative peptide inhibitor at time zero to a growing culture in liquid medium at 37° C. Cells analyzed for CR binding when reached optical density (A₆₀₀) of approximately 1.2 absorbance units (AU).

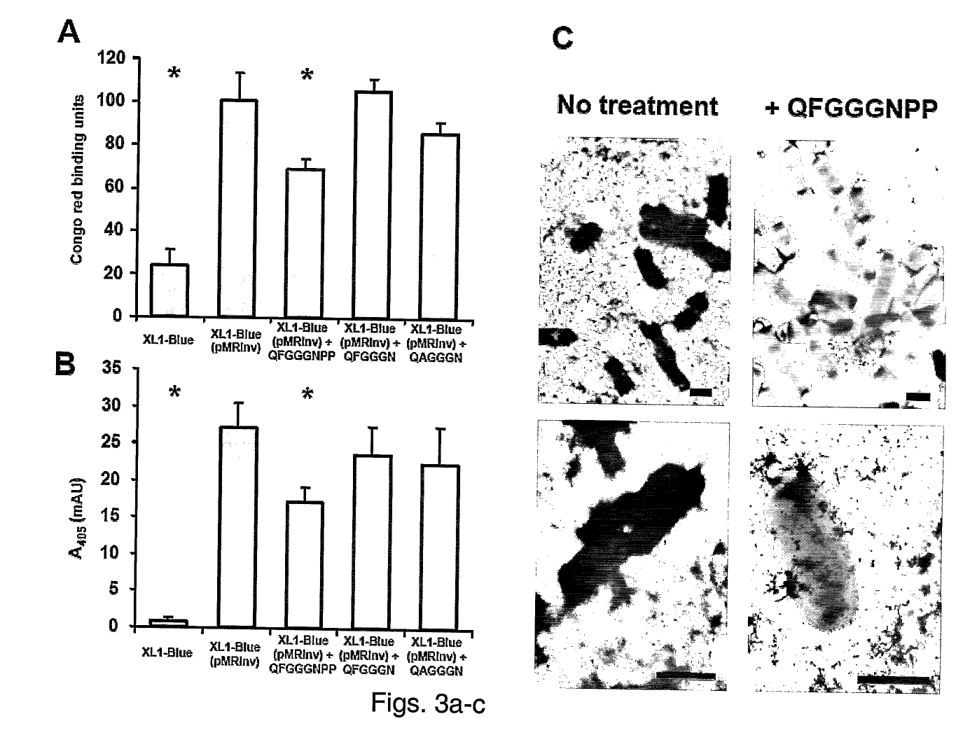

As expected, the curli-expressing bacteria displayed elevated levels of CR binding. However, in the presence of the peptide inhibitor, about 30% decrease in CR binding was observed (FIG. 3a), suggesting that the peptide interferes with curli polymerization. This was further substantiated in view of similar experiments that were performed in the presence of the QFGGGN (SEQ ID NO. 3) and QAGGGN (SEQ ID NO. 4) peptides that resulted in no significant difference in binding of CR in comparison to XL1-Blue(pMRInv) growth in its absence.

Fibronectin Binding

Further evidence for the effect of the peptide inhibitor came from the fibronectin binding assay. Curli expressing bacteria, liquid-grown in the presence or absence of the peptide inhibitor, were analyzed for adhesion to fibronectin coated microtiter wells. As seen in FIG. 3b, bacteria incubated in the presence of the peptide inhibitor were more easily washed from the wells, indicating they bound fibronectin less. To ensure that this actually stems from a decrease in curli polymerization rather than merely direct disruption the binding process, the oligopeptide repeats QFGGGN and QAGGGN were used exhibiting no significant effect on bacterial adhesion to fibronectin.

Morphology

Bacterial cultures were further inspected by electron microscopy in order to compare the morphology of the curli fimbriae produced with or without the peptide inhibitor. Curli expressing bacteria produced dense and developed fimbrial matrix surrounding the bacteria (FIG. 3c). This phenotype correlates with a high level of curli fiber expression (33). However, in the presence of the peptide inhibitor, the matrix surrounding the bacteria did not develop into a comparable morphology, but instead remained sparse. It is suggested that this difference, which correlates with CR and fibronectin binding properties (shown above), is associated with curli formation and solely derives from the presence of the peptide inhibitor.

It should be noted that sampling the bacterial culture following longer period of incubation, revealed that the bacteria eventually (matter of hours) overcame the inhibition and produced similar matrix surrounding the untreated bacteria. This is probably due to imbalance between the curlin subunits, continuously produced and secreted by the bacteria, and the constant concentration of the peptide. For future utilization of the concept for the development of effective therapeutic agents, proteolytic stable building blocks (such as D-amino acids) should be used.

Bacteria Uptake

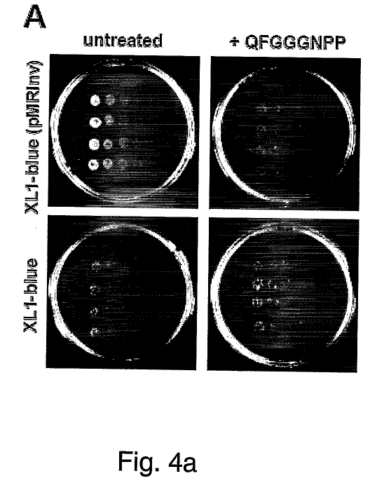

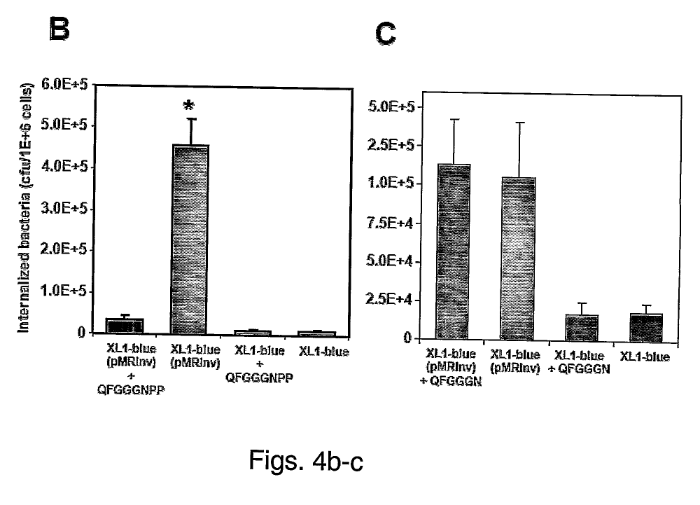

The effect of the peptide inhibitor on in vitro uptake of E. coli by eukaryotic cells, was assayed. For that purpose, an antibiotic protection assay was effected using HEK293 cell-line. This assay is based on the fact that internalized bacteria gain invulnerability to certain antibiotic drugs that do not cross the cellular membrane of the host, such as gentamicin. Mid-exponential phase bacteria, grown either in the presence or absence of the peptide inhibitor, were incubated with the cell-line for three hours at 37° C. Following exposure to the antibiotic gentamicin, eukaryotic cells were analyzed for bacteria internalization rate by the determination of the colony forming units (cfu) of the surviving bacteria (FIG. 4a). While the amount of internalized E. coli harboring the cosmid was significantly much higher than E. coli lacking it, in the presence of peptide inhibitor their amount was considerably decreased (FIG. 4b). To rule out the possibility that this result is not specific to the inhibitor sequence, another invasion assay was performed, which verified that QFGGGN peptide did not interfere with bacterial invasion process (FIG. 4c). As curli expression and invasion into host cells were shown to correlate in independent studies (33-35), it is suggested that the present result stems from significant reduction in curli formation.

### CONCLUSIONS

Taken together, using three independent assays reflecting different properties of the curli fimbriae, the ability of a short peptide, based on conserved short repeats within the CsgA (and AgfA) sequence, to inhibit curli fibril formation was significantly demonstrated. In view of the above results, it is likely that these repeats play a central role in the recognition process of the curlin subunits in the procedure of curli fimbriae polymerization by self-assembly.

The results presented herein and previous studies on prionic proteins (7-15) suggest that the occurrence of oligopeptide repeats has an essential role in self-assembly mechanism. Although evolving from different evolutionary roots, all protein repeats display notable similarity in their chemical nature. It is now suggested that yeast prion, animal prion and bacteria curli represent a unique case of a convergence of independently evolved chemical strategies into one common self-assembly module. Hence, the repeat module technique may represent an optimal molecular machinery to efficiently mediate specific self-assembly.

The formation of typical amyloid by E. coli may also facilitate the search for therapeutic agent to treat amyloid disease. As amyloid fibrils formation by non-homologous protein appears to share common characteristic features, and small molecule amyloid inhibitors (such as Congo red or polyphenol catechins) show cross-reactivity, curli-related amyloid formation may serve as a model system to study potential amyloid inhibitors. The low-cost and reproducibility of the bacterial amyloid formation may overcome limitation set by the high cost and seeding variability of synthetic amyloidogenic polypeptide and allow high throughput screen of candidate inhibitors.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

### REFERENCES CITED BY NUMERALS

### Other References are Cites in Complete

1. Rochet, J. C. & Lansbury, P. T. Jr. (2000). Amyloid fibrillogenesis: themes and variations. Curr. Opin. Struct. Biol. 10, 60-68.
2. Serpell, L. C. (2000). Alzheimer's amyloid fibrils: structure and assembly. Biochim. Biophys. Acta. 1502, 16-30.
3. Dobson, C. M. (2003). Protein folding and misfolding. Nature 426, 884-890.
4. Chapman, M. R., Robinson, L. S., Pinkner, J. S., Roth, R., Heuser, J., Hammar, M., Normark, S. & Hulgren, S. J. (2002). Role of Escherichia coli curli operons in directing amyloid fiber formation. Science 295, 851-855.
5. Elliot, M. A., Karoonuthaisiri, N., Huang, J., Bibb, M. J., Cohen, S, N., Kao, C. M. & Buttner, M. J. (2003). The chaplins: a family of hydrophobic cell-surface proteins involved in aerial mycelium formation in Streptomyces coelicolor. Genes Dev. 17, 1727-1740.
6. Berson, J. F., Theos, A. C., Harper, D. C., Tenza, D., Raposo, G. & Marks, M. S. (2003). Proprotein convertase cleavage liberates a fibrillogenic fragment of a resident glycoprotein to initiate melanosome biogenesis. J. Cell. Biol. 161, 521-533.
7. Tuite, M. F. & Cox, B. S. (2003). Propagation of yeast prions. Nat. Rev. Mol. Cell. Biol. 4, 878-890.
8. Serio, T. R. & Lindquist, S. L. (2000). Protein-only inheritance in yeast: something to get [PSI⁺]-ched about. Trends Cell. Biol. 10, 98-105.
9. DePace, A. H., Santoso, A., Hillner, P. & Weissman, J. S. (1998). A critical role for amino-terminal glutamine/asparagines repeats in the formation and propagation of a yeast prion. Cell 93, 1241-1252.
10. King, C. Y., Tittmann, P., Gross, H., Gebert, R., Aebi, M. & Wuthrich, K. (1997). Prion-inducing domain 2-114 of yeast Sup35 protein transforms in vitro into amyloid-like filaments. Proc. Natl. Acad. Sci. USA 94, 6618-6622.
11. True, H. L., Berlin, I. & Lindquist, S. L. (2004). Epigenetic regulation of translation reveals hidden genetic variation to produce complex traits. Nature 431, 184-187.
12. Schätzl, H. M., Da Costa, M., Taylor, L., Cohen, F. E. & Prusiner, S. B. (1995). Prion protein gene variation among primates. J. Mol. Biol. 245, 362-374.
13. Goldfarb, L. G., Brown, P., McCombie, W. R., Goldgaber, D., Swergold, G. D., Wills, P. R., Cervenakova, L., Baron, H., Gibbs, C. J. Jr. & Gajdusek, D. C. (1991). Transmissible familial Creutzfeldt-Jakob disease associated with five, seven, and eight extra octapeptide coding repeats in the PRNP gene. Proc. Natl. Acad. Sci. USA 88, 10926-10930.
14. Flechsig, E., Shmerling, D., Hegyi, I., Raeber, A. J., Fischer, M., Cozzio, A., von Mering, C., Aguzzi, A. & Weissmann, C. (2000). Prion protein devoid of the octapeptide repeat region restores susceptibility to scrapie in PrP knockout mice. Neuron 27, 399-408.
15. Liu, J. J. & Lindquist, S. (1999). Oligopeptide-repeat expansions modulate ‘protein-only’ inheritance in yeast. Nature 400, 573-576.
16. Gazit, E. (2002). Global analysis of tandem aromatic octapeptide repeats: the significance of the aromatic-glycine motif. Bioinformatics 18, 880-883.
17. Collinson, S. K., Parker, J. M., Hodges, R. S. & Kay, W. W. (1999). Structural predictions of AgfA, the insoluble fimbrial subunit of Salmonella thin aggregative fimbriae. J. Mol. Biol. 290, 741-756.
18. Perutz, M. (1994). Polar zippers: their role in human disease. Protein Sci. 3, 1629-1637.
19. Aggeli, A., Bell, M., Boden, N., Keen, J. N., Knowles, P. F., McLeish, T. C., Pitkeathly, M. & Radford S. E. (1997). Responsive gels formed by the spontaneous self-assembly of peptides into polymeric beta-sheet tapes. Nature 20, 259-262.
20. Azriel, R. & Gazit, E. (2001). Analysis of the minimal amyloid-forming fragment of the islet amyloid polypeptide. An experimental support for the key role of the phenylalanine residue in amyloid formation. J. Biol. Chem. 276, 34156-34161.
21. Gazit, E. (2002). A possible role for □-stacking in the self-assembly of amyloid fibrils. FASEB J 16, 77-83.
22. Reches, M. & Gazit, E. (2003). Casting metal nanowires within discrete self-assembled peptide nanotubes. Science 300, 625-627.
23. Jones, S., Manning, J., Kad, N. M. & Radford, S. E. (2003). Amyloid-forming peptides from beta2-microglobulin-Insights into the mechanism of fibril formation in vitro. J. Mol. Biol. 10, 249-257.
24. Makin, O, S., Atkins, E., Sikorski, P., Johansson, J. & Serpell, L. C. (2005). Molecular basis for amyloid fibril formation and stability. Proc. Natl. Acad. Sci. USA (in press).
25. Viles, J. H., Cohen, F. H., Prusiner, S. B., Goodin, D. B., Wright, P. E. & Dyson, H. J. (1999). Copper binding to the prion protein: structural implications of four identical cooperative binding sites. Proc. Natl. Acad. Sci. USA 96, 2042-2047.
26. Burns, C. S., Aronoff-Spencer, E., Legname, G., Prusiner, S. B., Antholine, W. E., Gerfen, G. J., Peisach, J. & Millhauser, G. L. (2003). Copper coordination in the full-length, recombinant prion protein. Biochemistry 42, 6794-6803.
27. Soto, C., Sigurdsson, E. M., Morelli, L., Kumar, R. A., Castano, E. M. & Frangione, B. (1998). Beta-sheet breaker peptides inhibit fibrillogenesis in a rat brain model of amyloidosis: Implications for Alzheimer's therapy. Nat. Med. 4, 822-826.
28. Bieler, S. & Soto, C. (2004). Beta-sheet breakers for Alzheimer's disease therapy. Curr. Drug. Targets 5, 553-558.
29. Gilead, S. & Gazit, E. (2004). Inhibition of amyloid fibril formation by peptide analogues modified with alpha-aminoisobutyric acid. Angew. Chem. Int. Ed. Engl. 43, 4041-4044.
30. Loferer, H., Hammar, M. & Normark, S. (1997). Availability of the fibre subunit CsgA and the nucleator protein CsgB during assembly of fibronectin-binding curli is limited by the intracellular concentration of the novel lipoprotein CsgG. Mol. Microbiol. 26, 11-23.
31. Hammar, M., Arnqvist, A., Bian, Z., Olsen, A. & Normark, S. (1995). Expression of two csg operons is required for production of fibronectin- and Congo-red binding curli polymers in Escherichia coli K-12. Mol. Microbiol. 18, 661-670.
32. Olsen, A., Jonsson, A. & Normark, S. (1989). Fibronectin binding mediated by a novel class of surface organelles on Escherichia coli. Nature 338, 652-655.
33. Gophna, U., Barlev, M., Seijffers, R., Oelschlager, T. A., Hacker, J., Ron, E. Z. (2001). Curli fibers mediate internalization of Escherichia coli by eukaryotic cells. Infect. Immun. 69, 2659-2665.
34. Gophna, U., Oelschlager, T. A., Hacker, J. & Ron, E. Z. (2002). Role of fibronectin in curli-mediated internalization. FEMS Microbiol. Lett. 212, 55-58.
35. Uhlich, G. A., Keen, J. E. & Elder, R. O. (2002). Variations in the csgD promoter of Escherichia coli O157:H7 associated with increased virulence in mice and increased invasion of HEp-2 cells. Infect. Immun. 70, 395-399.

1. A method of preventing or treating a pathogen infection in a subject, the method comprising administering to a subject in need thereof a therapeutically effective amount of an anti amyloid agent, thereby treating or preventing the pathogen infection in the subject.

2. Use of an anti-amyloid agent for the manufacture of a medicament identified for preventing or treating a pathogen infection in a subject.

3. A method of typing a pathogen, the method comprising monitoring an alteration in growth and/or infectivity of the pathogen in the presence of an anti-amyloid agent, thereby typing the pathogen.

4. A method of identifying an anti-amyloid agent, the method comprising:
(a) contacting molecules with an amyloid forming pathogen: and
(b) identifying at least one molecule of said molecules capable of altering amyloid formation of said amyloid forming pathogen, thereby identifying the anti-amyloid agent.

5. A medical device comprising an anti-amyloid agent attached thereto.

6. The medical device of claim 5, wherein the medical device is an intracorporeal device.

7. The medical device of claim 5, wherein the medical device is an extracorporeal device.

8. The method and use of claim 1, wherein said pathogen infection comprises a bacterial infection.

9. The method and use of claim 1, wherein said pathogen infection comprises a fungi infection.

10. The methods, use and medical device of claim 1, wherein said anti-amyloid agent is a proteinaceous agent.

11. The methods, use and medical device of claim 10, wherein said proteinaceous agent is a peptide agent.

12. The methods, use and medical device of claim 11, wherein said peptide agent comprises an amino acid sequence selected from the group consisting of SEQ ID NO: 9, 10 and 11.

13. The methods, use and medical device of claim 11, wherein said peptide agent comprises an amino acid sequence X-Y or Y-X, wherein X is an aromatic amino acid and Y is any amino acid other than glycine, the peptide being at least 2 and no more than 15 amino acids in length.

14. The methods, use and medical device of claim 12, wherein at least one amino acid of said amino acid sequence of the peptide is a D stereoisomer.

15. The methods, use and medical device of claim 12, wherein at least one amino acid of said amino acid sequence of the peptide is an L stereoisomer.

16. The methods, use and medical device of claim 12, wherein Y is a polar uncharged amino acid selected from the group consisting of serine, threonine, asparagine, glutamine and natural derivatives thereof.

17. The methods, use and medical device of any of claim 12, wherein Y is a β-sheet breaker amino acid.

18. The methods, use and medical device of claim 17, wherein said β-sheet breaker amino acid is a naturally occurring amino acid.

19. The methods, use and medical device of claim 18, wherein said naturally occurring amino acid is selected from the group consisting of proline, aspartic acid, glutamic acid, glycine, lysine and serine.

20. The methods, use and medical device of claim 17, wherein said β-sheet breaker amino acid is a synthetic amino acid.

21. The methods, use and medical device of claim 20, wherein said synthetic amino acid is a Cα-methylated amino acid.

22. The methods, use and medical device of claim 21, wherein said Cα-methylated amino acid is α-aminoisobutyric acid.

23. The methods, use and medical device of claim 11, wherein the peptide is a linear or cyclic peptide.

24. The methods, use and medical device of claim 12, wherein the peptide is two amino acids in length and Y is a β-sheet breaker amino acid.

25. The methods, use and medical device of claim 12, wherein the peptide is 3 amino acids in length, whereas Y is an aromatic amino acid and an amino acid residue attached to said amino acid sequence X-Y or Y-X is a β-sheet breaker amino acid.

26. The methods, use and medical device of claim 25, wherein said β-sheet breaker amino acid is at a C-terminus of the peptide.

27. The methods, use and medical device of claim 12, wherein the peptide is at least 4 amino acids in length and includes at least two serine residues at a C-terminus thereof.

28. The methods, use and medical device of any of claim 12, wherein the peptide is at least 3 amino acids in length and includes a thiolated amino acid at an N-terminus thereof.

29. The methods, use and medical device of claim 12, wherein the peptide is at least 3 amino acids in length and whereas at least one of said amino acids of the peptide other than X-Y is a β-sheet breaker amino acid.

30. The methods, use and medical device of claim 12, wherein the peptide is at least 3 amino acids in length and whereas at least one of said amino acids of the peptide is a positively charged amino acid and at least one of said amino acids of the peptide is a negatively charged amino acid.

31. The methods, use and medical device of claim 1, wherein said anti-amyloid agent is a non-proteinaceous agent.

32. The methods, use and medical device of claim 31, wherein said non-proteinaceous agent comprises a compound having the general Formula I: 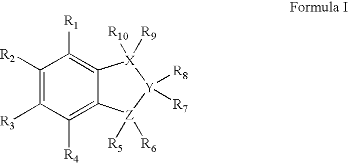
a pharmaceutically acceptable salt thereof or a prodrug thereof,
wherein:
X, Y and Z are each independently selected from the group consisting of carbon, oxygen, sulfur, CR₁₁R₁₂ or R₁₃R₁₄C—CR₁₅R₁₆, provided that at least one of X, Y and Z is oxygen or sulfur; and
R₁-R₁₆ are each independently selected from the group consisting of hydrogen, lone pair electrons, hydroxy, alkyl, cycloalkyl, phenyl, alkoxyphenyl, thioalkoxyphenyl, aryloxyphenyl, thioaryloxyphenyl, carboxyphenyl, thiocarboxyphenyl, phenol, hydroxyphenol, dihydroxyphenol, aryl, alkenyl, alkynyl, heteroaryl, heteroalicyclic, halo, alkoxy, aryloxy, thiohydroxy, thioalkoxy, thioaryloxy, C-carboxy, O-carboxy, thiocarboxy, carbonyl, oxo, thiocarbonyl, sulfinyl, and sulfonyl, or absent, or, alternatively, at least two of R₁-R₄ and/or at least two of R₅-R₁₆ form at least one five- or six-membered aromatic, heteroaromatic, alicyclic or heteroalicyclic ring,
whereas:
at least one of R₁-R₄ is selected from the group consisting of hydroxy, thiohydroxy, alkoxy, thioalkoxy, aryloxy, thioaryloxy, carboxy and thiocarboxy; and/or
at least one of R₅-R₁₆ comprises phenol, alkoxyphenyl, thioalkoxyphenyl, aryloxyphenyl, thioaryloxyphenyl, carboxyphenyl, thiocarboxyphenyl, hydroxyphenol, and dihydroxyphenol,
with the proviso that the compound is not any one of catechin, epicatechin, gallocatechin gallate, epigallocatechin gallate, epigallocatechin, and epicatechin gallate,
for the manufacture of a medicament identified for the treatment of amyloid-associated diseases.

33. The methods, use and medical device of claim 32, wherein:
X is carbon;
Y is R₁₃R₁₄C—CR₁₅R₁₆; and
Z is oxygen.

34. The methods, use and medical device of claim 33, wherein:
R₉ is oxo; and
R₁₀ is absent.

35. The methods, use and medical device of claim 33, wherein at least one of R₁₃-R₁₆ is selected from the group consisting of alkoxyphenyl, thioalkoxyphenyl, aryloxyphenyl, thioaryloxyphenyl, carboxyphenyl, thiocarboxyphenyl, phenol, hydroxyphenol and dihydroxyphenol.

36. The methods, use and medical device of claim 35, wherein each of R₁ and R₃ is hydroxy.

37. The methods, use and medical device of claim 33, wherein at least one of R₁₃-R₁₆ is alkyl.

38. The methods, use and medical device of claim 32, wherein:
X is carbon;
Y is oxygen;
Z is carbon or sulfur; and
at least one of R₅ and R₆ is oxo.

39. The methods, use and medical device of claim 34, wherein at least one of R₉ and R₁₀ is selected from the group consisting of alkoxyphenyl, thioalkoxyphenyl, aryloxyphenyl, thioaryloxyphenyl, carboxyphenyl, thiocarboxyphenyl, phenol, hydroxyphenol and dihydroxyphenol.

40. The methods, use and medical device of claim 32, wherein said compound is selected from the group consisting of phenol red, dimethoxy phenol red, methoxy phenol red, diacetoxy phenol red, acetoxy phenol red, pyrocatechol violet, phenolphthaleine, catechin, epigallocatechin gallate, epicatechin gallate, epicatechin, epigallocatechin, eriodictyol, quercetin, procyanidin, hydroxyphenyl, tocopherol, and bromophenol red.

41. The methods, use and medical device of claim 31, wherein said non peptide agent comprises a compound having the general formula: 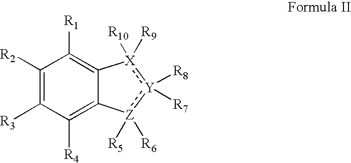
a pharmaceutically acceptable salt thereof, or a prodrug thereof,
wherein:
the dashed line denotes a double bond either between X and Y, or, between Y and Z;
X, Y and Z are each independently selected from the group consisting of carbon and nitrogen, whereas at least one of X, Y, and Z is nitrogen; and
R₁-R₁₀ are each independently selected from the group consisting of hydrogen, lone pair electrons, hydroxy, alkyl, cycloalkyl, phenyl, phenol, hydroxyphenol, dihydroxyphenol, aryl, alkenyl, alkynyl, heteroaryl, heteroalicyclic, halo, alkoxy, aryloxy, thiohydroxy, thioalkoxy, thioaryloxy, C-carboxy, O-carboxy, thiocarboxy, carbonyl, oxo, thiocarbonyl, sulfinyl, and sulfonyl, or absent, or, alternatively, at least two of R₁-R₁₀ form at least one five- or six-membered aromatic, heteroaromatic, alicyclic or heteroalicyclic ring,
thereby treating the amyloid associated disease in the subject.

42. The methods, use and medical device of claim 41, wherein:
each of X and Y is carbon; and
Z is nitrogen.

43. The methods, use and medical device of claim 42, wherein said double bond is between X and Y.

44. The methods, use and medical device of claim 41, wherein at least one of R₁-R₁₀ comprises a hydroxy group.

45. The methods, use and medical device of claim 43, wherein at least one of R₁-R₁₀ comprises a hydroxy group.

46. The methods, use and medical device of claim 45, wherein at least one of R₁ and R₉ comprises a hydroxy group.

47. The methods, use and medical device of claim 45, wherein at least one of R₁ and R₉ is a hydroxy group.

48. The methods, use and medical device of claim 47, wherein each of R₂-R₅ and R₇ is hydrogen and R₆, R₈ and R₁₀ are absent.

49. The methods, use and medical device of claim 48, wherein R₁ is hydrogen and R₉ is a hydroxy group.

50. The methods, use and medical device of claim 48, wherein R₁ is a hydroxy group and R₉ is hydrogen.

51. The methods, use and medical device of claim 45, wherein at least one of R₁-R₁₀ is a hydroxyalkyl.

52. The methods, use and medical device of claim 51, wherein at least one of R₇ and R₉ is a hydroxyalkyl.

53. The methods, use and medical device of claim 52, wherein each of R₁-R₅ is hydrogen and R₆, R₈ and R₁₀ are absent.

54. The methods, use and medical device of claim 52, wherein said hydroxyalkyl is hydroxymethyl.

55. The methods, use and medical device of claim 54, wherein R₇ is hydrogen and R₉ is said hydroxymethyl.

56. The methods, use and medical device of claim 55, wherein each of R₁-R₅ is hydrogen and R₆, R₈ and R₁₀ are absent.

57. The methods, use and medical device of claim 52, wherein each of R₇ and R₉ is a hydroxyalkyl.

58. The methods, use and medical device of claim 10, wherein said non-proteinaceous agent is a non-steroidal anti-inflammatory drug.

59. The methods, use and medical device of claim 10, wherein said non-proteinaceous agent is selected from the group consisting of nicotine, acridine, acridine orange, methylene blue, congo red, thioflavin-T and tetracycline.


SEQUENCE LISTING (11 sequences)

SEQ ID NO: 1 - protein, 9 aa, Artificial sequence
  Synthetic peptide
PQGGYQQYN

SEQ ID NO: 2 - protein, 8 aa, Artificial sequence
  Synthetic peptide
PHGGGWGQ

SEQ ID NO: 3 - protein, 6 aa, Artificial sequence
  Synthetic peptide
QFGGGN

SEQ ID NO: 4 - protein, 6 aa, Artificial sequence
  Synthetic peptide
QAGGGN

SEQ ID NO: 5 - protein, 6 aa, Artificial sequence
  Synthetic peptide
QHGGGN

SEQ ID NO: 6 - protein, 6 aa, Artificial sequence
  Synthetic peptide
NHQGGG

SEQ ID NO: 7 - protein, 6 aa, Artificial sequence
  Synthetic peptide
HGQGNG

SEQ ID NO: 8 - protein, 6 aa, Artificial sequence
  Synthetic peptide
GQNHGG

SEQ ID NO: 9 - protein, 8 aa, Artificial sequence
  Synthetic peptide
PQFGGGNP

SEQ ID NO: 10 - protein, 10 aa, Artificial sequence
  Synthetic peptide
PPQFGGGNPP

SEQ ID NO: 11 - protein, 8 aa, Artificial sequence
  Synthetic peptide
QFGGGNPP